(19) United States
(12) Patent Application Publication
(10) Pub. No.: US 2026/0207847 A1
(43) Pub. Date: Jul. 23, 2026

(54) THERAPEUTIC AGENT DELIVERY DEVICE WITH SYRINGE CARRIER

(71) Applicant: Eli Lilly and Company, Indianapolis, IN (US)

(72) Inventors: Jeremy Steven CULMER, Parma, OH (US); Kevin Harrison DUFFY, Pittsboro, IN (US); David Arthur HOLLEY, Lancaster, OH (US); Michael Lewis MARSHALL, Lewis Center, OH (US); Jake Michael PYZZA, Delaware, OH (US)

(21) Appl. No.: 19/134,546

(22) PCT Filed: Dec. 4, 2023
(86) PCT No.: PCT/US2023/082218

§ 371 Date: May 30, 2025

Related U.S. Application Data
(60) Provisional application No. 63/386,337, filed on Dec. 7, 2022.

(51) Int. Cl. A61M 5/20; A61M 5/32

(52) U.S. Cl. CPC A61M 5/2033; A61M 5/3204; A61M 5/3234; A61M 2005/208

(57) ABSTRACT

A therapeutic agent delivery device includes a reusable portion and a syringe carrier. The reusable portion includes a first housing and a cam ring rotatably movable within the first housing. The syringe carrier includes a second housing having a protrusion that enters a securing track of the cam ring upon insertion of the syringe carrier into the reusable portion, a first portion rotatably coupled to the second housing, a syringe assembly being movable between a stowed and a deployed configuration, and a base cap including a first surface engageable with the first portion. A first rotation of the cam ring rotates a securing portion of the securing track to receive the protrusion to lock the syringe carrier to the reusable portion and rotates the first portion to disengage the first surface of the base cap from the first portion to unlock the base cap from the first portion.

### FIELD

The present disclosure pertains to therapeutic agent delivery devices with syringe carriers, and, in particular, to a portable therapeutic agent delivery device such as an injector pen with a novel syringe carrier.

### BACKGROUND

Patients suffering from a number of different diseases frequently must inject themselves with medication. To allow a person to conveniently and accurately self-administer medicine, a variety of devices broadly known as injector pens have been developed. Generally, these devices are equipped with a cartridge including a piston and one or more doses of liquid medication. A drive member, extending from within a base of the injector pen and operably connected with typically more rearward mechanisms of the pen that control drive member motion, is movable forward to advance the piston in the cartridge in such a manner to dispense the contained medication from an outlet at the opposite cartridge end, typically through a needle that penetrates a stopper at that opposite end. In disposable pens, after a pen has been used and exhausted the supply of medication within the cartridge, the entire pen is discarded by a user, who may then begin using a replacement pen. In reusable pens, after a pen has been used and exhausted the supply of medication within the cartridge, the pen is disassembled, the spent cartridge is replaced with a fresh cartridge, and the pen is reassembled for its subsequent use.

It would be desirable to provide an injector pen with improved features, such as motorized locking and unlocking of the cartridge to and from the pen, automatic unlocking of a base cap of the cartridge, single use lockout to prevent reuse of the cartridge, and automatic ejection of the cartridge from the pen.

### SUMMARY

According to an embodiment of the present disclosure, a therapeutic agent delivery device includes a reusable portion and a syringe carrier. The reusable portion includes a first housing, a drive mechanism and a cam ring that is rotatably movable in the first housing by the drive mechanism. The syringe carrier includes a second housing with a first protrusion that enters a securing track of the cam ring upon insertion of the syringe carrier into the reusable portion. The syringe carrier also includes a first portion rotatably coupled to the second housing, a syringe assembly and a base cap. The syringe assembly includes a barrel containing a therapeutic agent, a needle and a needle shield detachably coupled to the barrel. The syringe assembly is movable between a stowed configuration and a deployed configuration wherein the needle extends from the second housing. The base cap includes a first surface for engaging the first portion to lock the base cap to the first portion. A first rotation of the cam ring rotates a securing portion of the securing track to receive the first protrusion to lock the syringe carrier to the reusable portion and rotates the first portion to disengage the first surface of the base cap from the first portion to unlock the base cap from the first portion.

According to another embodiment of the present disclosure, a therapeutic agent deliver device includes a reusable portion and a syringe carrier. The reusable portion includes a first housing, a drive mechanism and a cam ring rotatably movable in the first housing by the drive mechanism. The syringe carrier includes a second housing, a lockout spacer, an inject stop, a retraction spring, a syringe assembly and a base cap. The second housing includes a first protrusion and a second protrusion. The first protrusion enters a securing track of the cam ring upon insertion of the syringe carrier into the reusable portion. The lockout spacer includes a pair of third protrusions and is movable from an initial state to a single use lockout state. The inject stop includes a pair of control recesses for receiving the pair of third protrusions, and a pair notches. The retraction spring is connected to bias the lockout spacer for rotation toward the single use lockout state. The syringe assembly includes a barrel containing a therapeutic agent, a needle, and a needle shield detachably coupled to the barrel. The syringe assembly is movable between a stowed configuration and a deployed configuration wherein the needle extends from the second housing. The base cap includes a pair of tabs configured to engage the pair of notches to lock the base cap to the inject stop. A first rotation of the cam ring rotates a securing portion of the securing track to receive the first protrusion to prevent removal of the syringe carrier. The first rotation further rotates the inject stop to place the syringe carrier in a first configuration wherein the pair of tabs disengage from the pair of notches to unlock the base cap for removal, and the pair of third protrusions engage upper stop surfaces of the pair of control recesses to prevent movement of the syringe assembly toward the deployed configuration as the base cap is removed to detach the needle shield from the barrel. A second rotation of the cam ring rotates the inject stop to place the syringe carrier in a second configuration wherein the pair of third protrusions disengage from the upper stop surfaces to permit movement of the syringe assembly to the deployed configuration for delivery of the therapeutic agent. During movement of the syringe assembly to the stowed configuration after delivery of the therapeutic agent, the retraction spring rotates the lockout spacer to the single use lockout state wherein the pair of third protrusions engage the upper stop surfaces.

According to still another embodiment of the present disclosure, a method for delivering a therapeutic agent using a delivery device including a reusable portion and a syringe carrier includes inserting the syringe carrier into a housing of the reusable portion such that a first protrusion of the syringe carrier enters a securing track of a cam ring of the reusable portion, activating a drive mechanism to cause a first rotation of the cam ring, the first rotation rotating a securing portion of the securing track to receive the first protrusion to lock the syringe carrier to the reusable portion, the first rotation further causing rotation of an inject stop of the syringe carrier to disengage an engagement surface of a base cap of the syringe carrier to unlock the base cap from the inject stop, and activating the drive mechanism to cause a second rotation of the cam ring, the second rotation rotating the inject stop to disengage a second protrusion of a lockout spacer from a stop surface of the inject stop to permit movement of a syringe assembly of the syringe carrier to move to a deployed configuration for delivery of the therapeutic agent. During movement of the syringe assembly to a stowed configuration after delivery of the therapeutic agent, a retraction spring of the syringe carrier rotates the lockout spacer to a single use lockout state wherein the second protrusion engages the stop surface of the inject stop.

According to yet another embodiment of the present disclosure, a therapeutic agent delivery device includes a reusable portion and a syringe carrier. The reusable portion includes a first housing, a drive mechanism and a lock actuator movable in the first housing by the drive mechanism. The syringe carrier includes a carrier housing with a first carrier lock element coupled to a corresponding lock actuator element of the lock actuator upon insertion of the syringe carrier into the reusable portion, a first portion movably coupled to the carrier housing, a syringe assembly configured to hold a medication, a base cap including a first surface configured to engage with the first portion to couple the base cap to the first portion. A first movement of the lock actuator moves a securing portion of the lock actuator element to receive the first carrier lock element to lock the syringe carrier to the reusable portion, and moves the first portion to disengage the first surface of the base cap from the first portion to unlock the base cap from the first portion.

According to still another embodiment of the present disclosure, a syringe carrier includes a carrier housing, a lock portion movably coupled to the carrier housing, the lock portion having an injection lock and a carrier base cap lock, a syringe assembly configured to hold a medication, a lockout element axially fixed relative to the syringe assembly, the lockout element having a lockout lock. The lockout lock is coupled to the injection lock in a prevent state to prevent movement of the syringe assembly relative to the carrier housing. The syringe carrier also includes a base cap having a base cap lock coupled to the carrier base cap lock in a first configuration, and the base cap lock is configured to be decoupled from the carrier base cap lock in a second configuration, thereby permitting removal of the base cap relative to syringe assembly. In response to a first movement of the lockout element the base cap lock and the carrier base cap lock in the first configuration is transitioned to the second configuration. In response to a second movement of the lockout element, relative movement between the lockout lock and the injection lock decouples the lockout lock and the injection lock from the prevent state.

### BRIEF DESCRIPTION OF THE DRAWINGS

The above-mentioned and other advantages and objects of this disclosure, and the manner of attaining them, will become more apparent, and the disclosure itself will be better understood, by reference to the following description of embodiments of the invention taken in conjunction with the accompanying drawings, wherein:

FIG. 1 is a top perspective view of a therapeutic agent delivery device according to an embodiment of the present disclosure;

FIG. 2 is a bottom perspective view of the therapeutic agent delivery device of FIG. 1 with a disposable portion is shown detached from a reusable portion;

FIG. 3 is a transverse sectional view of the therapeutic agent delivery device along line 3-3 of FIG. 1 with a syringe assembly is shown in a stowed configuration;

FIG. 4 is a transverse sectional view of a distal end of the therapeutic agent delivery device of FIG. 1 with the syringe assembly is shown in a deployed configuration;

FIG. 5 is a schematic representation of an electronics assembly of the therapeutic agent delivery device of FIG. 1;

FIG. 6 is a detail transverse sectional view of a proximal end of the therapeutic agent delivery device within line 6 of FIG. 3;

FIG. 7 is a cross sectional view of the proximal end of the therapeutic agent delivery device along line 7-7 of FIG. 1;

FIG. 8 is a cross sectional view of the proximal end of the therapeutic agent delivery device along line 8-8 of FIG. 1;

FIG. 9 is a perspective view of a therapeutic agent delivery mechanism of the therapeutic agent delivery device of FIG. 1;

FIG. 10 is a detail transverse sectional view of the therapeutic agent delivery device within line 10 of FIG. 3;

FIG. 11 is a perspective view of a therapeutic agent delivery device according to another embodiment of the present disclosure;

FIG. 12 is a side view of the therapeutic agent delivery device of FIG. 11;

FIG. 13 is a side view of the therapeutic agent delivery device of FIG. 11 with a disposable portion removed from the reusable portion;

FIG. 14 is an exploded perspective view of the disposable portion of FIG. 13;

FIGS. 15 through 18 are side views of the therapeutic agent delivery device of FIG. 11 in various configurations;

FIG. 19 is a perspective view of portions of the therapeutic agent delivery device of FIG. 11 and corresponding to the configuration depicted in FIG. 15;

FIG. 20 is a perspective view of portions of the therapeutic agent delivery device of FIG. 11 and corresponding to the configuration depicted in FIG. 16;

FIG. 21 is an exploded side view of a therapeutic agent delivery device according to another embodiment of the present disclosure;

FIG. 22 is a perspective view of certain components of the therapeutic agent delivery device of FIG. 21;

FIGS. 23 and 24 are a detailed perspective views of certain components of the therapeutic agent delivery device of FIG. 21; and FIGS. 25 through 33 are side views of the therapeutic agent delivery device of FIG. 21 in various configurations.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present disclosure.

### DETAILED DESCRIPTION

Therapeutic agent delivery devices according to the present disclosure carry and dispense one or more therapeutic agents, which may also be referred to as medications or drugs. Such therapeutic agents may include, for example, epinephrine, anaesthetics, analgesics, steroids, insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, basal insulins, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, therapeutic agents for pain-related treatments, such as galcanzeumab or lasmiditan, or lebrikizumab and any therapeutic agent that is capable of delivery by the devices described herein. Devices according to the present disclosure may be operated in a manner generally as described herein by a user (for example, a healthcare professional, a caregiver, or another person) to deliver one or more medications to a patient (for example, another person or the user).

Any directional references used with respect to any of the Figures, such as right or left, up or down, or top or bottom, are intended for convenience of description, and do not limit the present disclosure or any of its components to any particular positional or spatial orientation. Additionally, any reference to rotation in a clockwise direction or a counterclockwise direction is simply illustrative. Any such rotation may be implemented in the reverse direction as that described herein.

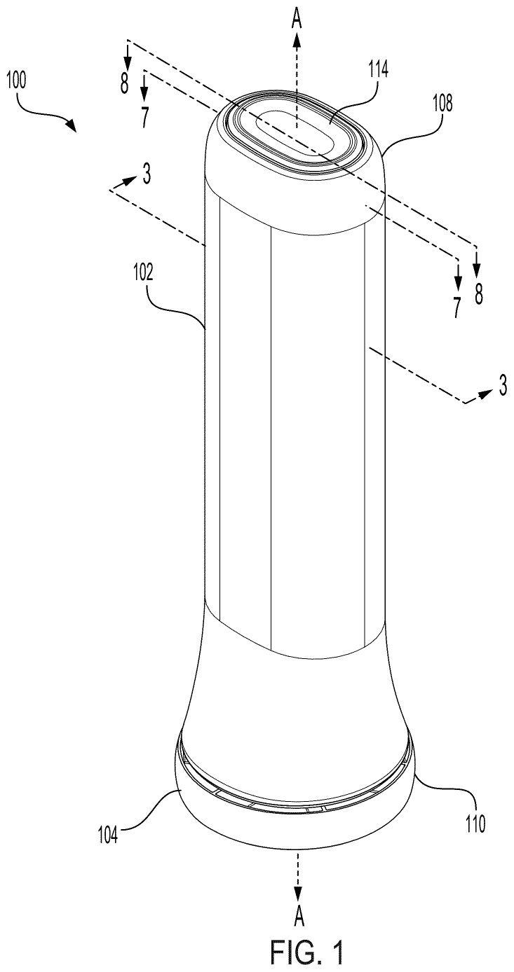

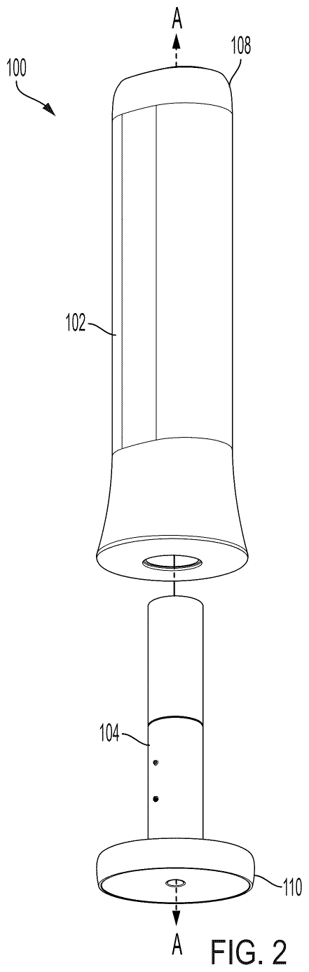

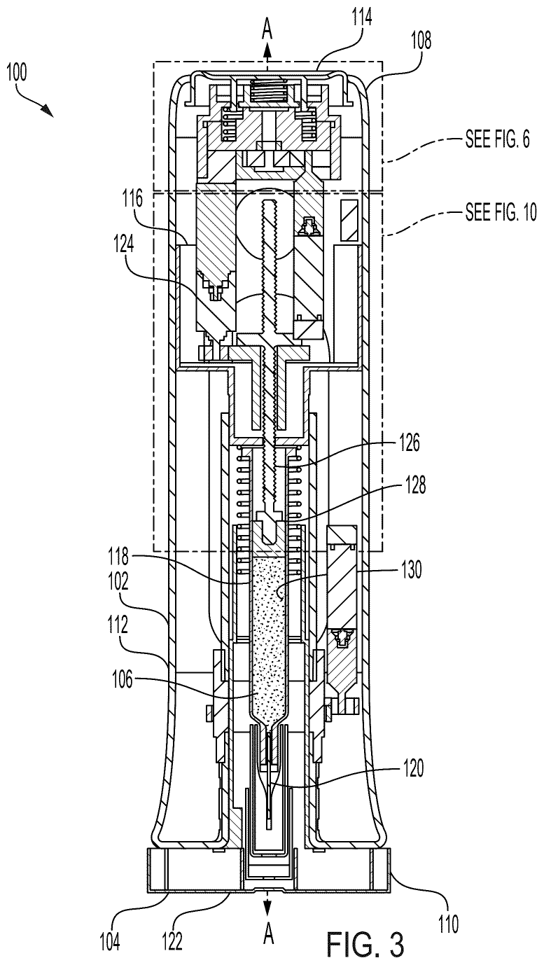

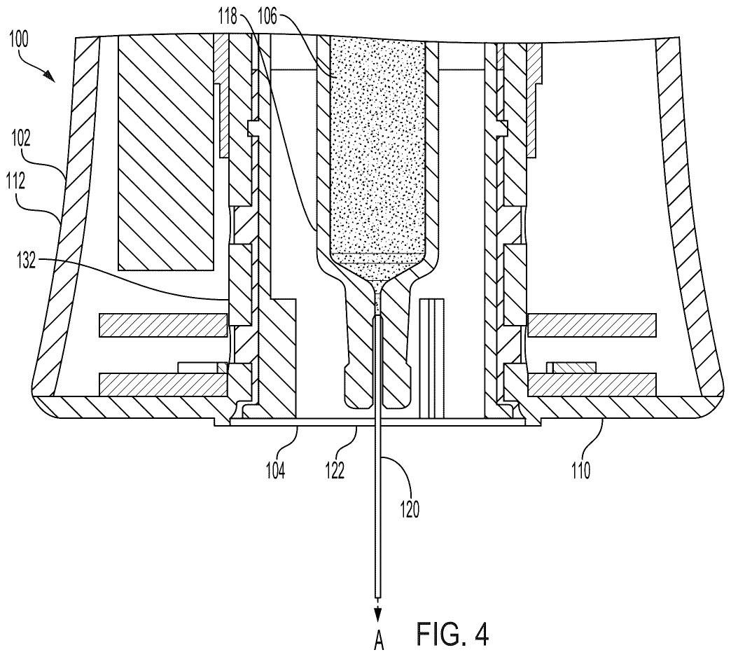

FIGS. 1-4 illustrate a therapeutic agent delivery device 100 according to an exemplary embodiment of the present disclosure. Illustratively, the therapeutic agent delivery device 100 has an injector pen-like shape, although other shapes may alternatively be used. The therapeutic agent delivery device 100 generally includes a reusable portion 102, which may also be referred to as a drive portion or a durable portion, and a disposable portion 104, which may also be referred to as a drug carrying portion, a cartridge or a syringe carrier. The reusable portion 102 facilitates delivery of a therapeutic agent 106 (FIGS. 3 and 4) from the disposable portion 104. In addition, the disposable portion 104 detachably couples to the reusable portion 102 such that after the therapeutic agent 106 has been delivered from the disposable portion 104, the disposable portion 104 may be detached from the reusable portion 102 and discarded. Another disposable portion (not shown for example, having the same or different features than the disposable portion 104) may then be attached to the reusable portion 102, and the therapeutic agent delivery device 100 is thereby ready for subsequent use.

The therapeutic agent delivery device 100 also includes a proximal end 108 and an opposite distal end 110. During use of the therapeutic agent delivery device 100, the proximal end 108 would be further from the patient and configured to be actuated by the user, and the distal end 110 would be closer to the patient and configured to deliver the therapeutic agent 106 to the patient. The therapeutic agent delivery device 100 also includes a longitudinal axis A extending between the proximal end 108 and the distal end 110. These and other features of the therapeutic agent delivery device 100 are described in further detail below.

With specific reference to the transverse sectional views of FIGS. 3 and 4, internal components and other features of the reusable portion 102 and the disposable portion 104 are illustrated. Generally, the reusable portion 102 includes a housing 112 that movably carries a user input 114 and a drive mechanism 116 (both shown in FIG. 3). The user input 114 is actuatable (for example, depressible) by a user to actuate the drive mechanism 116. The drive mechanism 116 thereby translates distally to drive a syringe assembly 118 of the disposable portion 104. More specifically, the drive mechanism 116 translatably drives the syringe assembly 118 from a stowed configuration (FIG. 3) to a deployed configuration (FIG. 4). In the stowed configuration, a needle 120 of the syringe assembly 118 is disposed proximally relative to a distal end 122 of the disposable portion 104. Stated another way, in the stowed configuration the needle 120 is retracted within the device 100. In the deployed configuration (FIG. 4), the needle 120 at least partially extends distally from the distal end 122 of the disposable portion 104. As a result, in the deployed configuration the needle 120 is configured to pierce the skin of a patient.

With specific reference to FIG. 3, the drive mechanism 116 of the reusable portion 102 also includes a plunger mechanism or therapeutic agent delivery mechanism 124. The therapeutic agent delivery mechanism 124 is actuatable to discharge the therapeutic agent 106 from the syringe assembly 118. More specifically, when the syringe assembly 118 is in the deployed configuration, the therapeutic agent delivery mechanism 124 is actuatable to distally translate a shaft or plunger 126 of the syringe assembly 118. The plunger 126 distally drives a piston 128 carried in a therapeutic agent-carrying passageway 130 or reservoir of the syringe assembly 118, which causes the therapeutic agent 106 to be discharged via the needle 120. In some embodiments, the delivery mechanism is configured to deliver a single dose from the syringe carrier prior to disposing of the syringe carrier. This single dose may be a fixed dose pre-set at manufacturing or a single dose that can vary based on the prescribing provider. In some embodiments, the delivery mechanism is configured to deliver a variable dose from the syringe carrier prior to disposing of the syringe carrier. The variably dose may be multiple fixed doses as priest at manufacturing or multiple variable doses that can vary based on the prescribing provider.

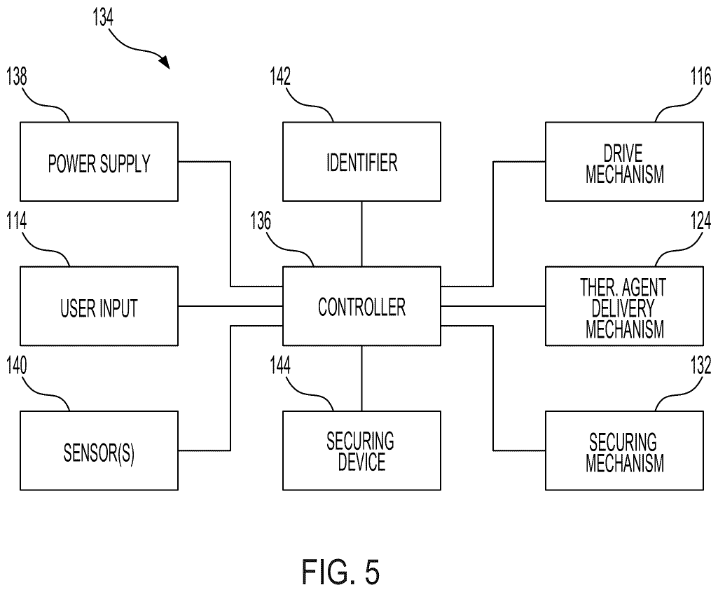

Referring to FIG. 5, in addition to the above components, the therapeutic agent delivery device 100 also includes an electronics assembly 134 that facilitates operating the device 100 in the manners described herein. The electronics assembly 134 includes an electronic controller 136 that is operatively coupled to and receives power from a power supply 138, such as a battery. The electronic controller 136 also operatively couples to the user input 114 and one or more sensors 140. As described in further detail below, the sensors 140 may sense, for example, actuation of components of the device 100, positions of components of the device 100 relative to each other, and/or the position of the device 100 relative to a patient. The controller 136 further operatively couples to the drive mechanism 116 (FIG. 3), the therapeutic agent delivery mechanism 124 (FIG. 3), and the securing mechanism 132 (FIG. 4).

In some embodiments and as described in further detail below, some components of the electronics assembly 134 are carried by the reusable portion 102 and some components are carried by the disposable portion 104 (both shown in FIGS. 1 and 4). For example, the disposable portion 104 may include an identifier 142 (for example, an RFID transmitter or EEPROM) to facilitate providing properties of the therapeutic agent 106 to the reusable portion 102. Such properties may include, for example, the type and/or volume of the therapeutic agent 106 carried by the syringe assembly 118. The reusable portion 102 may use the properties of the therapeutic agent 106 to determine, for example, if a patient associated with the reusable portion 102 is authorized to use, or has been prescribed, the therapeutic agent 106. As another example, a disposable portion may include securing device 144 that is operably coupled to the controller 136. The securing device 144 may initially inhibit the syringe assembly 118 from moving from the stowed configuration to the deployed configuration, and the controller 136 may actuate the securing device 144 to permit the syringe assembly 118 to move from the stowed configuration to the deployed configuration. In other embodiments, each of the components of the electronics assembly 134 is carried by the reusable portion 102. In some embodiments, the controller 136 is operatively coupled to one or more of the other components of the electronics assembly 134 by a wired connection. In some embodiments, the controller 136 is operatively coupled to one or more of the other components of the electronics assembly 134 by a wireless connection.

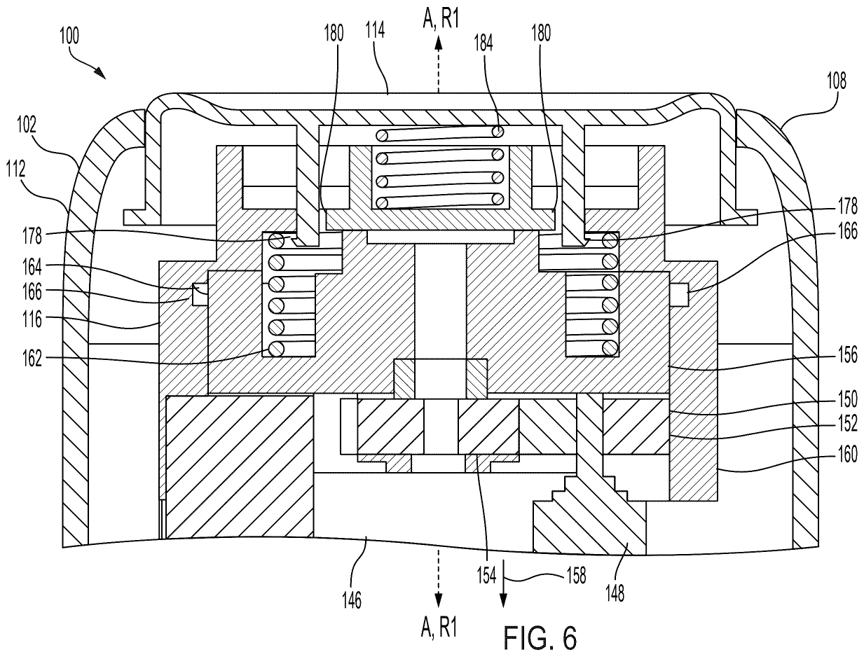

Referring now to FIGS. 6-8, the proximal end 108 of the device 100, specifically the user input 114 and a proximal portion of the drive mechanism 116, is shown in further detail. The drive mechanism 116 includes a carriage 146 (FIG. 6) that is translatably carried in the housing 112 of the reusable portion 102. The carriage 146 carries a first actuator 148 that operatively couples to the electronic controller 136 (FIG. 5). The first actuator 148 may be a rotary actuator, more specifically an electric motor, that drivably couples to a transmission or speed reducer. The actuator 148 drivably couples to a gear train 150, more specifically a first gear 152 that drivably couples to a second gear 154. The second gear 154 is fixed relative to a follower 156, and the follower 156 is rotatably carried by the carriage 146. As such, the carriage 146, the actuator 148, the gear train 150, and the follower 156 are translatable together within the housing 112 of the reusable portion 102. Illustratively, the carriage 146, the actuator 148, the gear train 150, and the follower 156 are translatable in a drive direction 158 (FIG. 6) that is substantially parallel to the longitudinal axis A of the device 100 (that is, parallel ±10 degrees).

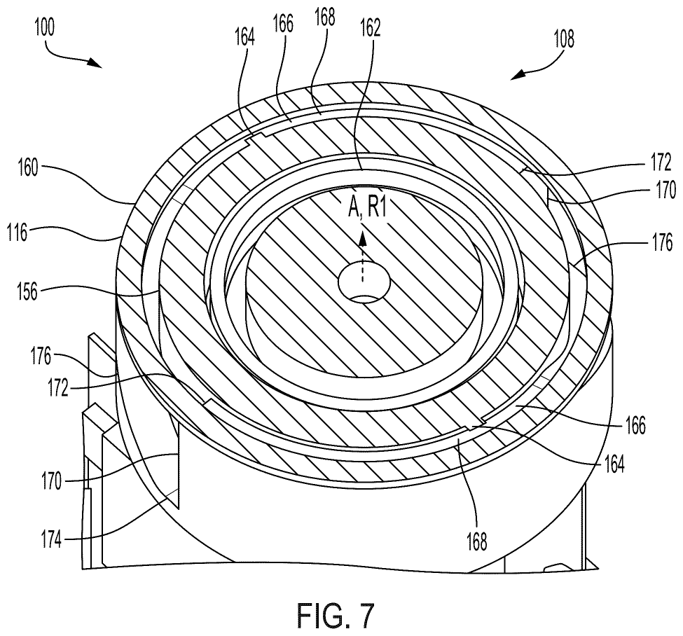

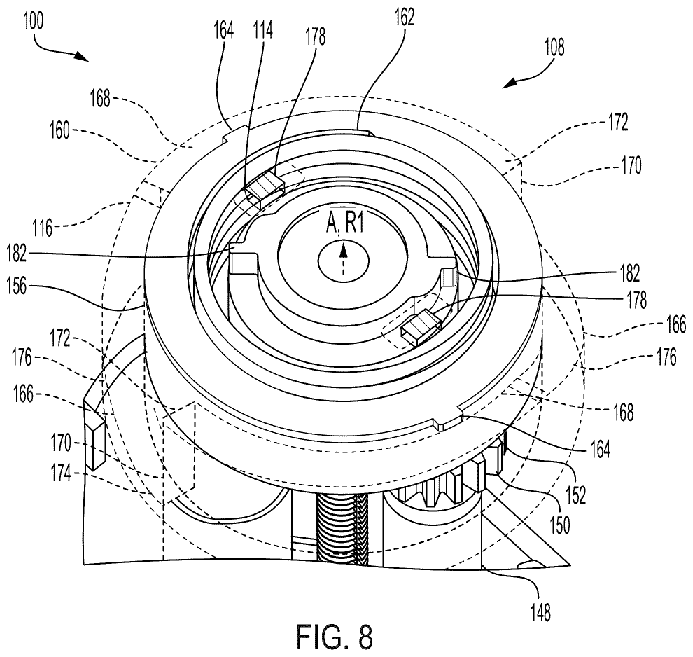

The follower 156 movably couples to a guide 160, and the guide 160 is fixed relative to the housing 112 of the reusable portion 102. A compression spring 162 urges the follower 156 distally and into engagement with the guide 160. Generally, the follower 156 and the guide 160 include features that facilitate translating the follower 156 relative to the guide 160 as the follower 156 rotates relative to the guide 160 about a rotation axis R1 that is substantially parallel to the longitudinal axis A of the device 100 (that is, parallel ±10 degrees). More specifically, the follower 156 includes two radially-outwardly extending protrusions 164 that move along an angular track 166, or generally proximally-facing wall, defined by the guide 160 as the follower 156 is rotated by the actuator 148. The protrusions 164 simultaneously move along two similar sections, or halves, of the track 166. Referring specifically to FIGS. 7 and 8, each half of the track 166 includes a plateau portion 168 that couples to a cliff portion 170 at an edge 172, a valley portion 174 coupled to the cliff portion 170 opposite the plateau portion 168, and a slope portion 176 coupled to the valley portion 174 opposite the cliff portion 170. Each slope portion 176 also couples to the plateau portion 168 of the other half of the track 166.

In the illustrated embodiment, the various portions of the track 166 are as follows. The cliff portions 170 are substantially parallel to the longitudinal axis A of the device 100 (that is, parallel ±10 degrees). The plateau portions 168 and the valley portions 174 are substantially perpendicular to the longitudinal axis A of the device 100 (that is, perpendicular ±10 degrees). The slope portions 176 extend helically relative to the longitudinal axis A of the device 100.

In other embodiments, the follower 156 and/or the guide 160 may have different forms. For example, the track 166 could have a different shape. More specifically, the track could include additional slope portions (not shown) instead of the cliff portions 170, and such slope portions could extend helically in the opposite directions as the slope portions 176. As another example, the follower 156 could include a different number of protrusions 164 and/or the guide 160 could include a track 166 with a different number of similar sections. As yet another example, the follower 156 could include a track 166 that movably receives one or more protrusions 164 formed on the guide 160.

With specific reference now to FIG. 6, the proximal end 108 of the device 100 also includes features for selectively inhibiting motion of the user input 114 relative to the housing 112 of the reusable portion 102 and, as a result, actuation of the user input 114. More specifically, the user input 114 includes snap hooks 178 that extend through openings 180 formed in the guide 160. The hooks 178 engage the guide 160 and hold the user input 114 in a depressed configuration relative to the housing 112. With specific reference now to FIGS. 6 and 8, the follower 156 includes legs 182 (FIG. 8) that engage and release the hooks 178 from the guide 160 as the follower 156 rotates relative to the guide 160. When the hooks 178 disengage the guide 160, a compression spring 184 expands and pushes the user input 114 to an elevated configuration relative to the housing 112. Further details regarding the motion and several configurations of the various components at the proximal end 108 of the device 100 are described in U.S. Provisional Application No. 63/234,955, Attorney Docket No. ELC-P22867-01-US, entitled THERAPEUTIC AGENT DELIVERY DEVICE INCLUDING DISPOSABLE AND REUSABLE PORTIONS, the entire contents of which are expressly incorporated herein by reference.

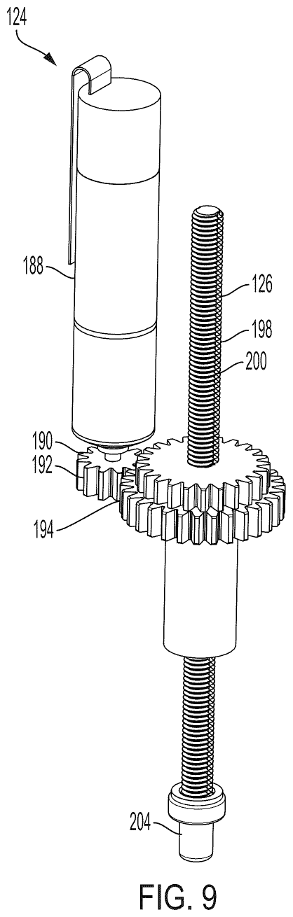

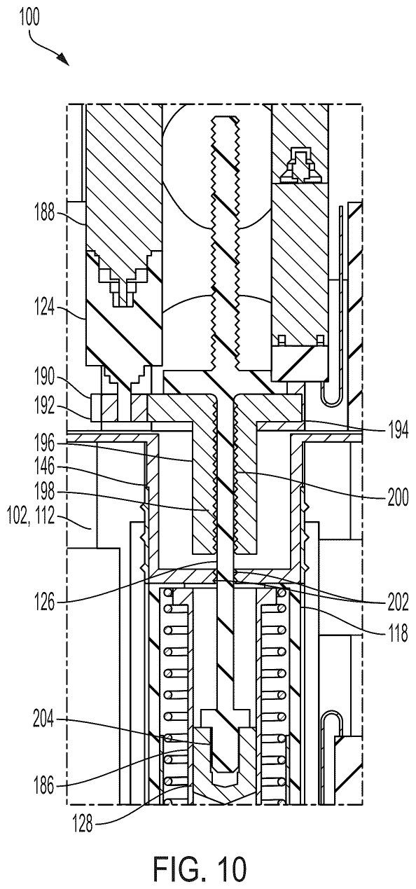

FIGS. 9 and 10 illustrate the therapeutic agent delivery mechanism 124 of the device 100. In the transverse sectional view of FIG. 10, the therapeutic agent delivery mechanism 124 is also illustrated adjacent other components of the device 100, such as the carriage 146, a syringe chamber 186, the piston 128.

With continued reference to FIGS. 9 and 10, the therapeutic agent delivery mechanism 124 is carried by and translates with the carriage 146 relative to the housing 112 of the reusable portion 102. The therapeutic agent delivery mechanism 124 includes a second actuator 188 that operatively couples to the electronic controller 136 (FIG. 5). The second actuator 188 may be a rotary actuator, more specifically an electric motor, that drivably couples to a transmission or speed reducer. The actuator 188 drivably couples to a gear train 190, more specifically a first gear 192 that drivably couples to a second gear 194. The second gear 194 includes internal threads 196 (FIG. 10) that couple to external threads 198 of the plunger 126. The plunger 126 is rotatably fixed but translatable relative to the carriage 146 (FIG. 10). Specifically, the plunger 126 couples to the carriage 146 via a key and slot interface, more specifically the plunger 126 includes external slots 200 that receive keys 202 (FIG. 10) formed on the carriage 146. The plunger 126 also includes a ram 204 for engaging the piston 128 of the syringe assembly 118.

With further reference to FIGS. 9 and 10, motion of the various components of the therapeutic agent delivery mechanism 124 is as follows. The actuator 188 is energized to rotatably drive the gear train 190 relative to the carriage 146. The plunger 126 thereby translates relative to the second gear 194 and the carriage 146. The plunger 126 distally pushes the piston 128 in the syringe chamber 186. As described above, such motion of the piston 128 causes the syringe assembly 118 to deliver the therapeutic agent from the needle 120.

With general reference to FIGS. 1-10, the controller 136 (FIG. 5) may actuate the drive mechanism 116 and the therapeutic agent delivery mechanism 124 sequentially upon detecting one or more conditions. More specifically, in some embodiments the drive mechanism 116 is actuated to move the syringe assembly 118 from the stowed configuration to the deployed configuration, and thereafter the therapeutic agent delivery mechanism 124 is actuated to drive the plunger 126 and the piston 128 and thereby deliver the therapeutic agent 106 from the needle 120. In these embodiments, the sensors 140 of the electronics assembly 134 may include a position sensor, such as an encoder (not shown) coupled to the actuator 148, for determining if the syringe assembly 118 is in the stowed configuration or the deployed configuration. Upon detecting that the syringe assembly 118 is in the deployed configuration, the therapeutic agent delivery mechanism 124 may be actuated to deliver the therapeutic agent 106 from the needle 120. After delivering the therapeutic agent 106, the drive mechanism 116 is actuated again to permit the syringe assembly 118 to move from the deployed configuration to the stowed configuration, and then the therapeutic agent delivery mechanism 124 is actuated again to retract the plunger 126 from the syringe assembly 118. More specifically, upon detecting that the syringe assembly 118 has returned to the stowed configuration, the therapeutic agent delivery mechanism 124 is actuated to retract the plunger 126 from the syringe assembly 118.

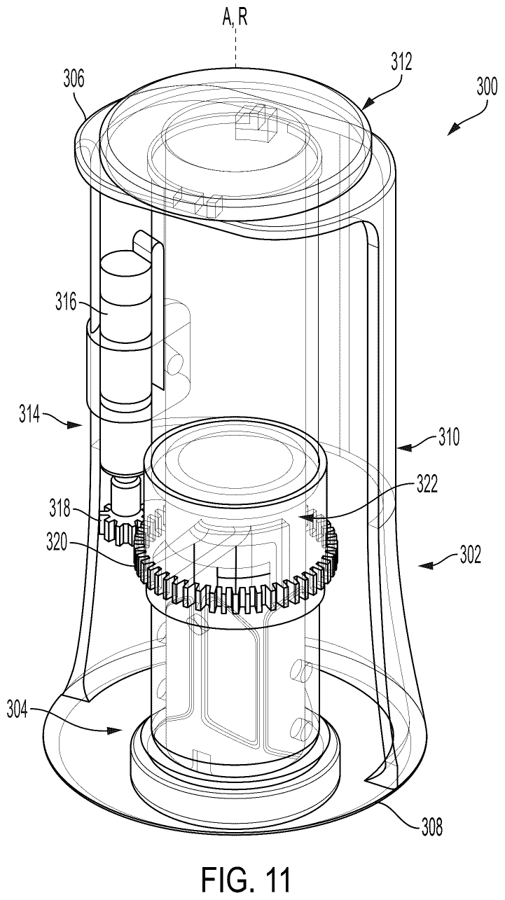

As shown in FIG. 11, a therapeutic agent delivery device 300 according to another exemplary embodiment of the present disclosure generally includes a reusable portion 302, which may also be referred to as a drive portion or a durable portion, and a disposable portion 304, which may also be referred to as a drug carrying portion, a cartridge, or a syringe carrier. The reusable portion 302 facilitates delivery of a therapeutic agent from the disposable portion 304 in a manner substantially as that described above with reference to reusable portion 102. The disposable portion 304 detachably couples to the reusable portion 302 such that after the therapeutic agent has been delivered, the used disposable portion 304 is automatically ejected from the reusable portion 302. Another disposable portion (not shown-for example, having the same or different features as disposable portion 304) may then be attached to the reusable portion 302, and the therapeutic agent delivery device 300 is then ready for a subsequent use.

The therapeutic agent delivery device 300 includes a proximal end 306 and an oppose distal end 308. During use of the therapeutic agent delivery device 300, the proximal end 306 is farther from the patient and configured to be actuated by a user, and the distal end 308 is closer to the patient and configured to deliver the therapeutic agent to the patient. The therapeutic agent delivery device 300 also includes a longitudinal axis A extending between the proximal end 306 and the distal end 308. These and other features of the therapeutic agent delivery device 300 are described in further detail below.

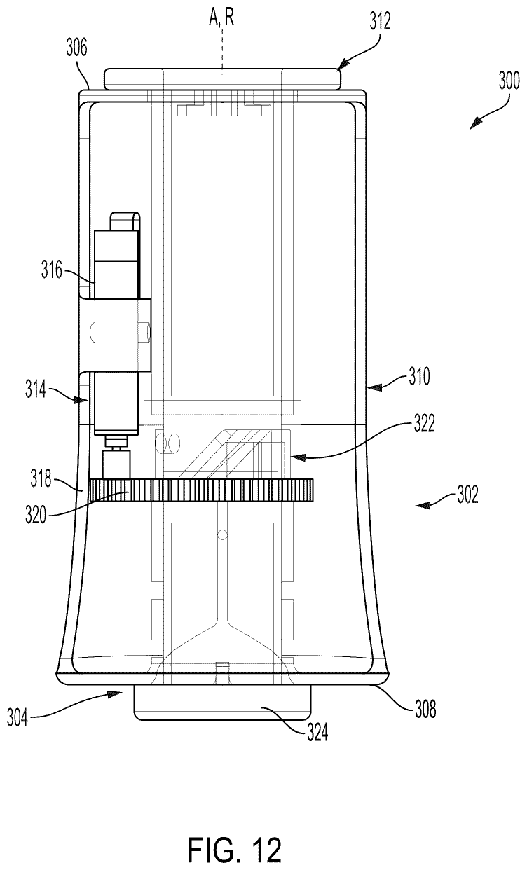

As shown in FIGS. 11 and 12, the reusable portion 302 includes a housing 310 including a user input 312 and a drive mechanism 314. The user input 312 is actuatable (for example, depressible) by a user to actuate the drive mechanism 314. In general, the drive mechanism 314 and the user input 312 of the reusable portion 302 may include components configured in a manner described above with reference to device 100 to cause a therapeutic agent to be forced through the needle into the patient. Additionally, the therapeutic agent delivery device 300 may also include an electronics assembly such as that described above with reference to device 100 to facilitate the operation of the various functions of the therapeutic agent delivery device 300 described herein.

Referring back to FIGS. 11 and 12, the drive mechanism 314 of the reusable portion 302 further includes an actuator 316 that operably couples to a controller 136 (FIG. 5). The actuator 316 may be a rotary actuator, more specifically an electric motor, that drivably couples to a transmission or speed reducer (not shown). The actuator 316 is coupled to a drive gear 318 that drivably engages a driven gear 320. The actuator 316 is operably coupled to a lock actuator that is movable relative to the housing 310 to lock the syringe carrier 304 to the reusable portion 302, and to unlock the base cap 324 from a lock portion of the syringe carrier 304. In one embodiment, the driven gear 320 is fixed relative to the lock actuator, also referred herein as a cam ring 322, which is rotatably disposed within the housing 310 of reusable portion 302. Generally, the cam ring 322 rotates relative to the housing 310 of the reusable portion 302 about a rotation axis R that is substantially parallel to the longitudinal axis A of the therapeutic agent delivery device 300 (i.e., parallel ±10 degrees). As is further described below, rotation of the cam ring 322 controls configurations of the disposable portion 304 throughout the process of delivering the therapeutic agent to the patient, from loading the disposable portion 304 into the reusable portion 302 to ejecting the used disposable portion 304 from the reusable portion 302.

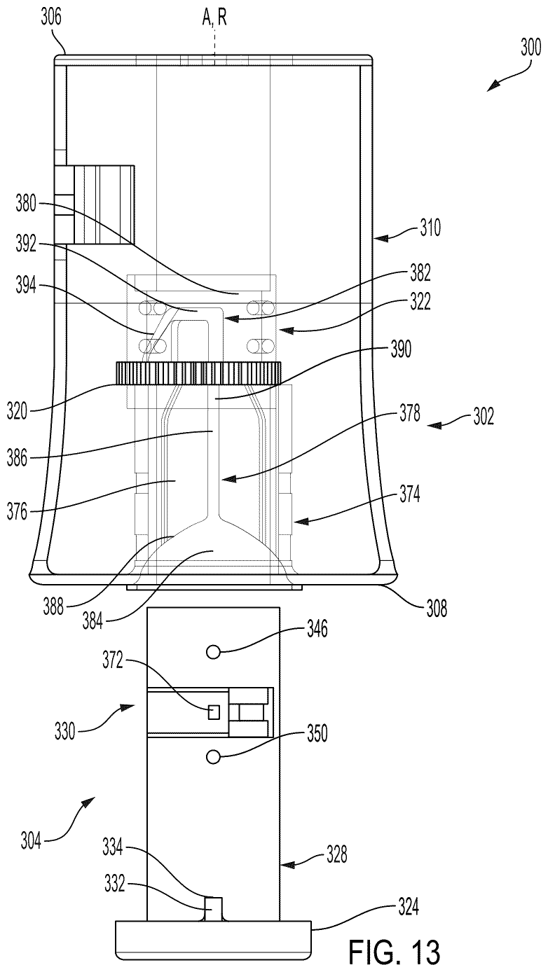

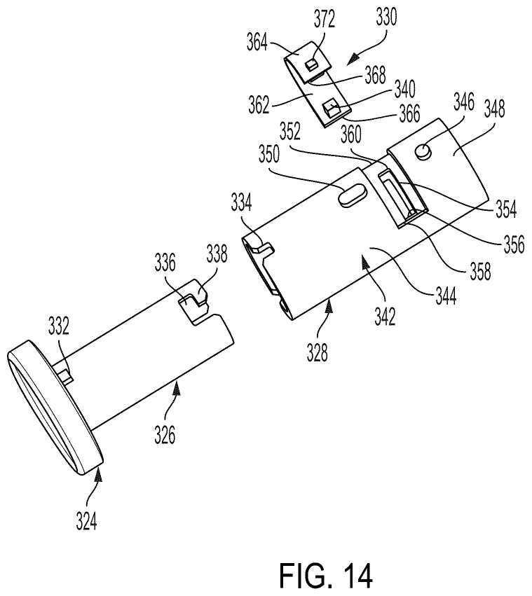

Referring now to FIGS. 13 and 14, the syringe carrier or disposable portion 304, which is depicted throughout this embodiment in simplified form omitting a variety of components, generally includes a base cap 324 coupled to a rigid needle shield (“RNS”) puller 326 which extends from the base cap 324, an outer housing 328 and a locking clip 330 (also referred to herein as “a first portion” or “a lock portion”). The base cap and the outer housing are configured to not rotate relative to one another at a first configuration. In one example, one of the base cap and the outer housing includes one or more tabs, and the other includes a corresponding key to receive the tab. In one embodiment, the base cap 324 includes a pair of tabs 332 which engage keys 334 formed into outer housing 328 when disposable portion 304 is assembled, thereby preventing rotation of base cap 324 and RNS puller 326 relative to outer housing 328. The base cap and the outer housing are configured to have a locked configuration. In one example, the locking clip 330 is configured to engage the RNS puller 326 through the wall of the outer housing 328 in a manner to lock the components together, and then capable of being released from engagement to permit the unlocking of the RNS puller. In one embodiment, the RNS puller 326 includes a pair of recess 336 (only one shown in FIG. 14) which each form an arm 338 which receives an inner protrusion 340 of the locking clip 330 to lock base cap 324 and RNS puller 326 to outer housing 328 until the locking clip 330 is rotated in the manner described below.

The outer housing 328 includes a cylindrical body 342 formed by a wall 344 with an inner diameter that is larger than an outer diameter of the RNS puller 326 such that the RNS puller 326 can be positioned within the outer housing 328.

The outer housing 328 may include guide features to facilitate alignment and positioning of the disposable portion 304 relative to the reusable portion 304. In one embodiment, the outer housing 328 further may include one or more syringe carrier lock element, such as, for example, a pair of upper protrusions 346 (only one shown in FIG. 14) formed on an outer surface 348 of outer housing 328 and a pair of lower protrusions 350 (only one shown in FIG. 14) formed on the outer surface 348. Each upper protrusion 346 is aligned with a lower protrusion 350 in the direction of the longitudinal axis A of the device 300. As is described in detail below, the upper protrusions 346 and the lower protrusions 350 interact with a lock actuator element or tracks formed on components of the reusable portion 302 to control movement of the disposable portion 304 from its insertion into its ejection from the reusable portion 302. It is understood that in other embodiments the track is formed along the disposable portion and the protrusions are defined by the reusable portion. The outer housing 328 further can include a clip location feature, such as, for example, a recess 352 formed into the outer surface 348 of the outer housing 328 which extends at least partially around the periphery of the outer housing 328. A pair of slots 354 are formed within the recess 352 and extend through the wall 344 of the outer housing 328. Each slot 354 includes a first end 356 disposed adjacent an end 358 of the recess 352 and second end 360 opposite the first end 356.

The locking clip 330 is formed in the shape of a semi-circle and includes an inner surface 362, an outer surface 364, a first end 366 and a second end 368. In one embodiment of the locking clip, protrusions may be defined along the interior and exterior of the body of the clip 330. For example, a pair of inner protrusions 340 (only one shown in FIG. 14) extend from the inner surface 362 of the locking clip 330 adjacent the first end 366 and the second end 368. A pair of outer protrusions 372 (only one shown in FIG. 14) extend from the outer surface 364 of the locking clip 330 adjacent the first end 366 and the second end 368. The width of the locking clip 330 corresponds substantially to the width of the recess 352 formed into the outer housing 328. The first end 366 of the locking clip 330 is radially spaced from the second end 368 by more than 180 degrees.

The locking clip 330 is attached to the outer housing 328 after the base cap 324 and RNS puller 326 are inserted into outer housing 328. The locking clip 330 is somewhat resilient such that the ends 366, 368 of the clip 330 flex away from one another as the clip 330 is inserted over the outer diameter of the recess 352 of the outer housing 328 and flex back toward one another when the clip 330 is fully seated within the recess 352. In other words, the locking clip 330 snaps over the outer housing 328 and into the recess 352. When the locking clip 330 is installed into the recess 352 of the outer housing 328, the inner protrusions 340 extend through the slots 354 formed through the wall 344 of the outer housing 328 and into the recesses 336 of the RNS puller 326. Specifically, the inner protrusions 340 are positioned under the arms 338 of the RNS puller 326 to prevent the RNS puller 326 from being removed from the outer housing 328.

The locking clip 330 is movable relative to the outer housing 328 and the RNS puller 326 to define an unlocked configuration. As is further described below, the locking clip 330 is rotatable about the rotation axis R within recess 352 of the outer housing 328 to cause the inner protrusions 340 of the locking clip 330 to rotate out from under the arms 338 of the RNS puller 326 to “unlock” the RNS puller 326 and permit its removal (along with RNS (not shown) of the syringe assembly (not shown)) from the reusable portion 302. It should be understood that the ends 356 of the slots 354 formed through the wall 344 of the outer housing 328 and the ends 358 of the recess 352 formed into the wall 344 are spaced apart from one another by substantially more than 180 degrees to permit rotational movement of the locking clip 330 about the rotation axis R within the recess 352.

Referring now to FIGS. 13 through 18, the cam ring 322 is carried within the housing 310 of the reusable portion 302 adjacent a distal cylinder 374, which is fixed within the housing 310. As such, the cam ring 322 rotates relative to the distal cylinder 374 about the rotation axis R. The cam ring 322 and the distal cylinder 374 include features that facilitate selectively securing the disposable portion 304 to the reusable portion 302. More specifically, an internal surface 376 of the distal cylinder 374 includes one or more leading tracks 378 (illustratively, two leading tracks 378—only one leading track 378 is visible in FIGS. 13 through 18), or slots, for receiving the upper protrusions 346 and the lower protrusions 350 formed onto the outer housing 328 of the disposable portion 304. The leading track 378 is configured to align the disposable portion 304 within the reusable portion 302 at a pre-defined angular position relative to one another. Similarly, an internal surface 380 of the cam ring 322 includes one or more securing tracks 382 (illustratively, two securing tracks 382—only one securing track 382 is shown in FIGS. 13 through 18), or slots (also referred herein as “a lock actuator element”, for receiving the upper protrusions 346 of the disposable portion 304. As is described in further detail below, the cam ring 322 rotates relative to the distal cylinder 374 to selectively align and misalign the securing tracks 382 with the leading tracks 378. The rotation permits and inhibits, respectively, the upper protrusions 346 of the disposable portion 304 to move between the leading tracks 378 and the securing tracks 382, which facilitates selectively securing the disposable portion 304 to the reusable portion 302 and ejecting the disposable portion 304 from the reusable portion 302.

With further reference to FIGS. 13 through 18, each leading track 378 of the distal cylinder 374 includes an inverted funnel shape. Each leading track 378 includes a tapering distal portion 384 and a relatively narrow proximal portion 386. Each distal portion 384 tapers in width proceeding proximally. More specifically, each distal portion 384 includes two oppositely helical extending walls 388 that couple to the proximal portion 386. As a result, the walls 388 are configured to direct the upper protrusions 346 of the disposable portion 304 toward the proximal portion 386 as the disposable portion 304 is coupled to the reusable portion 302. The proximal portion 386 of each leading track 378 may have a width that is slightly larger than the width of the upper protrusions 346. The proximal portion 386 of each leading track 378 may be substantially parallel to the longitudinal axis A (i.e., parallel ±10 degrees).

With specific reference to FIG. 13, each securing track 382 of the cam ring 322 includes an entry portion 390, a securing portion 392, and an egress portion 394. The entry portion 390 of each securing track 382 is selectively alignable with one the leading tracks 378 to facilitate receiving the upper protrusions 346 of the disposable portion 304 therefrom. The entry portion 390 of each securing track 382 may be substantially parallel to the longitudinal axis A (i.e., parallel ±10 degrees). As such, the entry portion 390 may be referred to as a longitudinal portion. Opposite the leading tracks 378, the entry portion 390 of each securing track 382 couples to the securing portion 392. The securing portion 392 may be substantially perpendicular to the longitudinal axis A (i.e., perpendicular ±10 degrees). As such, the securing portion 392 may be referred to as a transverse portion. Opposite the entry portion 390, the securing portion 392 of each securing track 382 couples to the egress portion 394. The egress portion 394 of each securing track 382 may extend helically relative to the longitudinal axis A and away from the securing portion 392. As such, the egress portion 394 may be referred to as a helical portion. The egress portion 394 is selectively alignable with one of the leading tracks 378 to facilitate transferring the upper protrusions 346 of the disposable portion 304 thereto.

Still referring to FIGS. 13 through 18, the motion and several configurations of the insertion and ejection of the disposable portion 304 into and from the reusable portion 302 are described below. Although the disposable portion 304 may remain in some of the configurations for certain periods of time, other configurations are shown for illustrative purposes, and the disposable portion 304 may simply transition through those configurations without remaining in them for a period of time. FIG. 13 illustrates a first or initial configuration of the disposable portion 304 wherein the disposable portion 304 is detached from the reusable portion 302 prior to use.

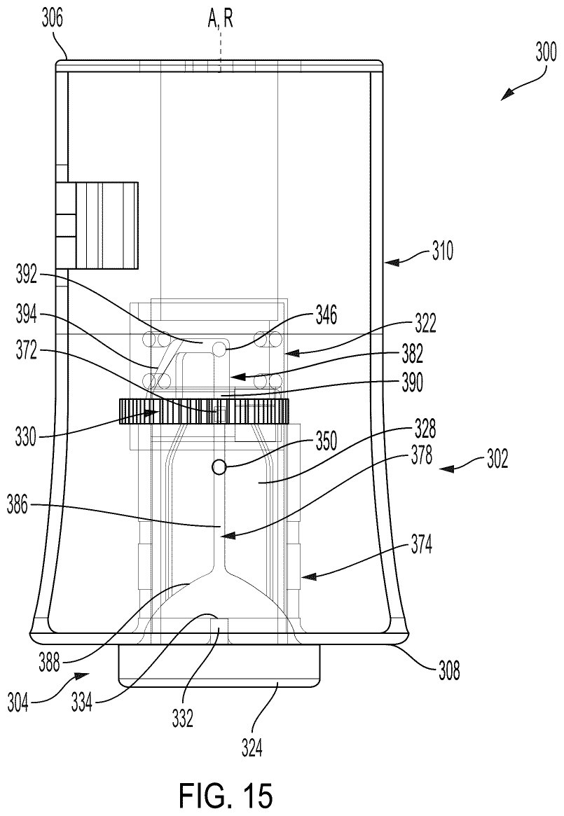

The disposable portion 304 is advanced proximally and toward the reusable portion 302 to arrive in a second configuration, as shown in FIG. 15. In the second configuration, the outer housing 328 of the disposable portion 304 is received in the distal cylinder 374 and the cam ring 322. In the second configuration, the upper protrusions 346 of the disposable portion 304 (one upper protrusion 346 being visible in FIG. 15) have passed through the leading tracks 378 of the distal cylinder 374 (one leading track 378 being visible in FIG. 15) and entered the entry portions 390 of the securing tracks 382 of the cam ring 322. Additionally, the outer protrusions 372 of the locking clip 330 have also entered the entry portions 390 of the securing tracks 382 of the cam ring 322. In the second configuration, the leading tracks 378 of the distal cylinder 374 are aligned with the entry portions 390 of the securing tracks 382 of the cam ring 322 (one entry portion 390 being visible in FIG. 15).

In the second configuration, the base cap 324 of the disposable portion 304 may abut the distal end 308 of the therapeutic agent delivery device 300. The lower protrusions 350 of the outer housing 328 of the disposable portion 304 (one lower protrusion 350 being visible in FIG. 15) have entered the proximal portions 386 of the leading tracks 378 of the distal cylinder 374.

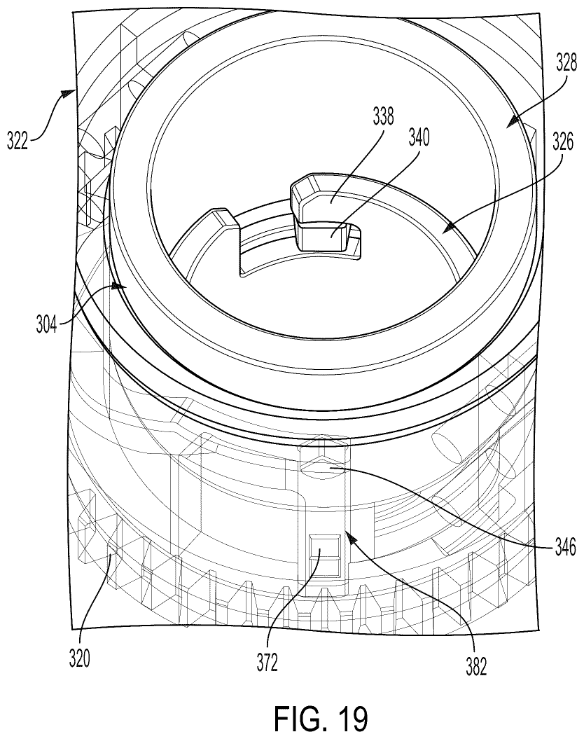

Additionally, as best shown in FIG. 19, as the disposable portion 304 is inserted into reusable portion 302, the inner protrusions 340 of the locking clip 330 remain positioned under the arms 338 of the RNS puller 326. Thus, in this configuration, the RNS puller 326 is locked to the disposable portion 304 and the disposable portion 304 is unlocked relative to the reusable portion 302 (i.e., not mechanically retained within the reusable portion 302). This second configuration is illustrated in both FIGS. 15 and 19.

Further action of the cam ring 322 is paused when the cam ring 322 is in the second configuration until the disposable portion 304 is identified by the controller 136, such as by reading an RFID tag attached to the disposable portion 304. Other identification methods are contemplated by the present disclosure, such as, for example, using an NFC label on the disposable portion 304 and an NFC sensor on the reusable portion 302. After the disposable portion 304 is identified, the controller 136 waits until the user is identified. More specifically, a user may press and hold a button (not shown) which may provide a signal to controller 136 through mechanical actuation of a switch or electrical actuation using capacitance detection or other suitable means. Alternatively, the user may be identified through voice identification or biometrics such as fingerprint identification. In other embodiments, no user identification is required. It should be noted that if the user is never identified or if the disposable portion 304 is determined not to match the expected identification, the user may be prompted to remove the disposable portion 304 from the reusable portion 302 via a display screen and/or an audible indicator (not shown). In such an event, the RNS puller 326 remains locked to the disposable portion 304.

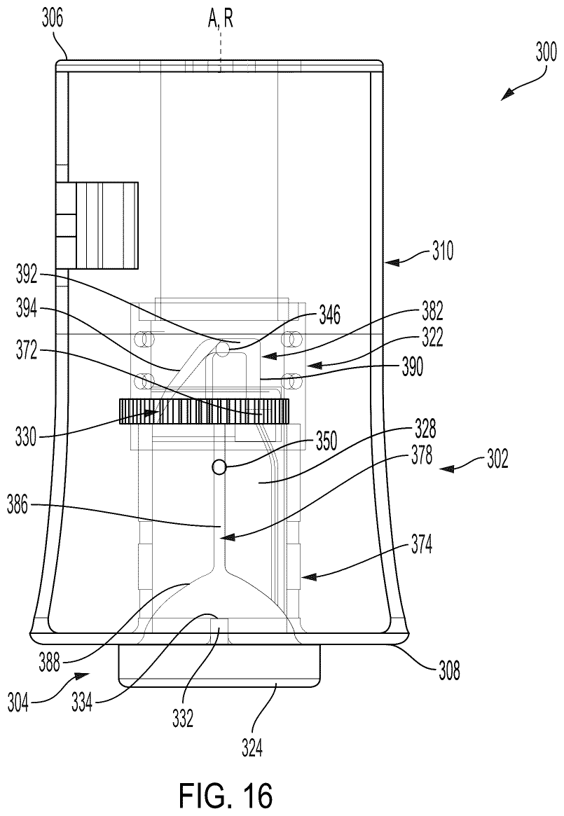

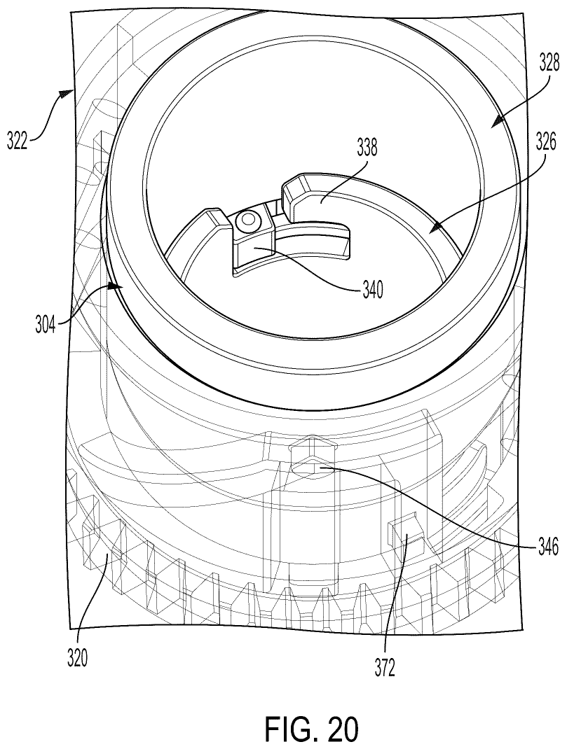

Referring to FIG. 12 and FIG. 16, after the disposable portion 304 and the user are identified and determined to be valid by the controller 136, the actuator 316 is energized to lock the disposable portion 304 to the reusable portion 302 and unlock the RNS puller 326 from the disposable portion 304 to permit its removal from the reusable portion 302, placing the disposable portion 304 in a third configuration. More specifically, the actuator 316 through gears 318 and 320 causes the cam ring 322 to rotate relative to the distal cylinder 374 (illustratively, in a counter clockwise direction as viewed from the proximal end 306 of the device 300). As the cam ring 322 rotates, the entry portions 390 of the securing tracks 382 of the cam ring 322 engage the outer protrusions 372 of the locking clip 330, which causes the locking clip 330 to rotate within the recess 352 (FIG. 14) of the outer housing 328 of the disposable portion 304. Engagement between the leading tracks 378 of the distal cylinder 374 and the lower protrusions 350 of the outer housing 328 of the disposable portion 304 prevent rotation of the outer housing 328. As best shown in FIG. 20, rotation of the locking clip 330 as a result of rotation of the cam ring 322 causes the inner protrusions 340 of the locking clip 330 to rotate out from under the arms 338 of the RNS puller 326, thereby unlocking the RNS puller 326 from the outer housing 328 of the disposable portion 304.

In alternative embodiments, the rotation of the locking clip 330 described above may be performed manually to unlock the RNS puller 326 from the outer housing 328 of the disposable portion 304. Any of a variety of different mechanisms for rotating locking clip 330 may be used, such as, for example, an outer ring may be movable on reusable portion 302 and configured to engage and rotate outer protrusions 372 of the locking clip as the outer ring is rotated. In other examples, the outer protrusions 372 may be accessible and manually movable by the user, or configured to engage a lever, slider or other mechanism that cooperates with the outer protrusions 372 to rotate the locking clip 330.

Additionally, as the cam ring 322 rotates, the upper protrusions 346 of the disposable portion 304 (one upper protrusion 346 being visible in FIG. 16) are positioned in the securing portions 392 of the securing tracks 382 of the cam ring 322 (one securing portion 392 and one securing track 382 being visible in FIG. 16). This inhibits axial detachment of the disposable portion 304 from the reusable portion 302 due to the securing portion 392 being engageable with the upper protrusion 392 and physically blocking the upper protrusion 346 from distal movement. Thus, rotation of the cam ring 322 places disposable portion 304 in the third configuration wherein the disposable portion 304 is locked in place within the reusable portion 302 by the securing portions 392 of the cam ring 322 and the RNS puller 326 is unlocked.

It should be understood, however, that in an alternative embodiment, the rotation of the cam ring 322 and the configuration of the disposable portion 304 may be modified such that a first rotation of the cam ring 322 positions the upper protrusions 346 of the disposable portion 304 within the securing portions 392 of the securing tracks 382 of the cam ring 322 to lock the disposable portion 304, and a second rotation of the cam ring 322 rotates the outer protrusions 372 of the locking clip 330 out from under the arms 338 of the RNS puller 326 to unlock the RNS puller 326 for removal.

In certain embodiments, the disposable portion 304 may remain in the third configuration until a user removes the RNS puller 326 and the base cap 324 of the disposable portion 304 from the reusable portion 302. This removal may be sensed by one of the sensors 140 of the reusable portion 302 and communicated to the controller 136. For example, the sensor 140 may be an electromechanical switch, a capacitance or magnetic field sensor, such as a Hall effect sensor, positioned in proximity to a corresponding metal or magnetic surface on the RNS puller 326 or the base cap 324 when the disposable portion 304 is fully inserted into the reusable portion 302. When the RNS puller 326 and the base cap 324 are removed from the reusable portion 302, the signal sensed by the sensor 140 changes in a manner that is interpreted by the controller 136 as corresponding to removal of the RNS puller 326 and the base cap 324. If the RNS puller 326 is not removed from the reusable portion 302, then the controller 136 may reverse the rotation direction of the actuator 316, thereby causing the cam ring 322 to return to the second configuration and indicate to the user that the disposable portion 304 should be removed by a message on a display or an audible message or otherwise of the device 100 and/or an external device, such as a smartphone.

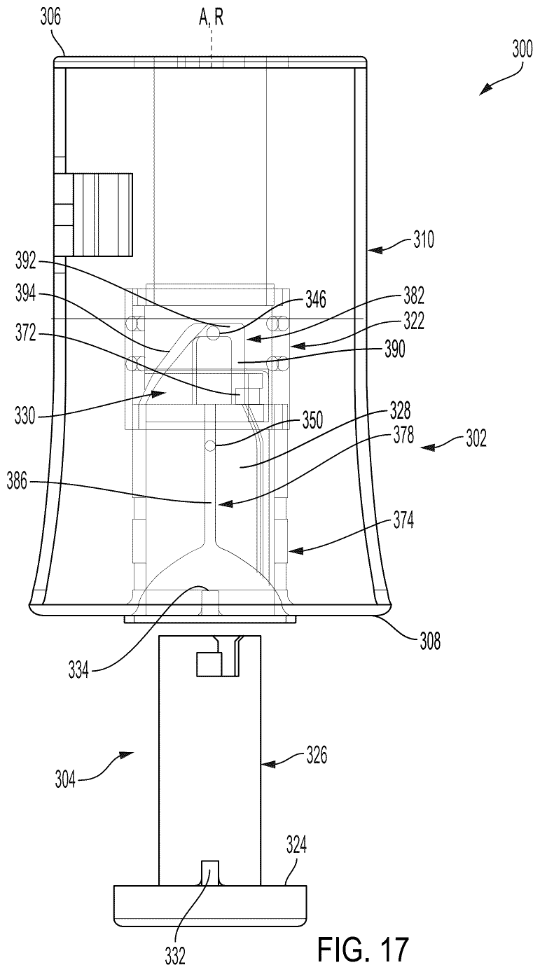

After the RNS puller 326 and the base cap 324 are removed from the reusable portion 302, the outer housing 328 (and internal components which are not shown) of the disposable portion 304 remains in place. This fourth configuration is illustrated in FIG. 17. When disposable portion 304 is in the fourth configuration, the controller 136 waits for a signal indicating that the distal end 308 of the reusable portion 302 is in contact with the skin of the patient. This signal may be provided by one or more sensors 140 disposed adjacent a lower surface of the distal end 308 of the reusable portion 302. For example, a skin sensor 140 may be a capacitance or other sensor configured to detect contact with the skin of the patient and provide a sensed signal to the controller 136 that changes with contact to indicate such contact. After contact with the patient's skin is detected, the user input 312 is enabled in the manner described herein. When the user actuates the user input 312, the drive mechanism 314 of the reusable portion 302 causes delivery of the therapeutic agent in the manner described herein. If the user input 312 is not actuated, or not actuated within a predetermined time limit (i.e., a dose of therapeutic agent is not delivered to the patient within the predetermined time limit), then the actuator 316 may cause the cam ring 322 to rotate to the fifth configuration described below to eject the disposable portion 304 containing therapeutic agent, but without the RNS puller 326 or the base cap 324 connected.

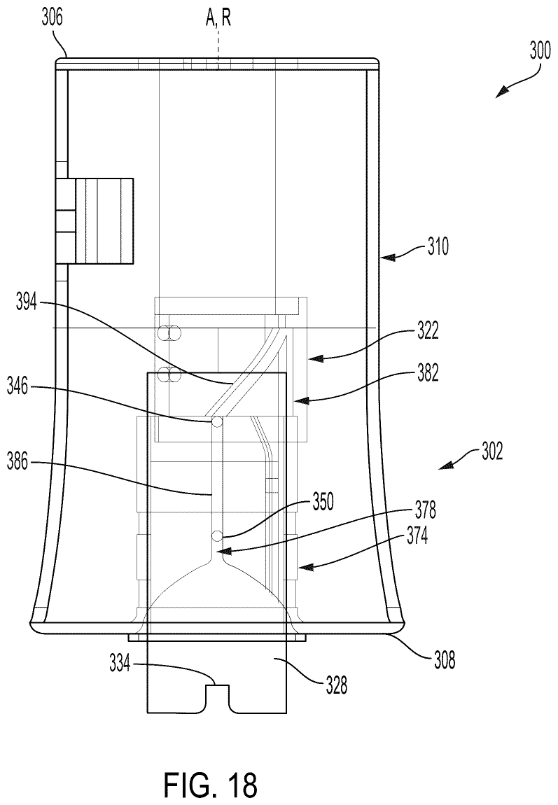

After the therapeutic agent is delivered to the patient, the cam ring 322 then rotates relative to the distal cylinder 374 (in the same direction-illustratively, in the counter-clockwise direction as viewed from the proximal end 306) such that the cam ring 322 moves to a fifth configuration depicted in FIG. 18. In the fifth configuration, the upper protrusions 346 of the outer housing 328 (one upper protrusion 346 being visible in FIG. 18) are disposed in the egress portions 394 of the securing tracks 382 of the cam ring 322 (one egress portion 394 and one securing track 382 being visible in FIG. 18). As a result, the cam ring 322 pushes the upper protrusions 346 and thus the disposable portion 304 distally relative to the reusable portion 302 to eject the disposable portion 304. In this fifth configuration, the egress portions 394 of the securing tracks 382 of the cam ring 322 (one egress portion 394 and one securing track 382 being visible in FIG. 18) are aligned with the leading tracks 378 of the distal cylinder 374 (one leading track 378 being visible in FIG. 18). Additionally, the upper protrusions 346 and the lower protrusions 350 of the disposable portion 304 are disposed in the leading tracks 378 of the distal cylinder 374. In certain embodiments, the user may be required to provide input such as by actuating a capacitive slider (not shown) to initiate the ejection sequence described above. In either case, the user may pull the disposable portion 304 distally to detach the disposable portion 304 from the reusable portion 302. At this stage, the controller 136 may wait for a signal from one or more sensors 140 indicating that the user has removed the disposable portion 304 from the reusable portion 302. The sensor 140 may also be used to indicate the presence of the disposable portion 304 when loaded into the reusable portion 302. For example, a sensor 140 disposed in the reusable portion 302 may detect the absence of the disposable portion 304 and provide a signal to the controller 136 indicating that the disposable portion 304 has been removed. The presence and/or absence of the disposable portion 304 may be sensed using an electromechanical switch, a capacitance or magnetic field sensor, such as a Hall effect sensor, an RF ID tag, or any other suitable sensing configuration. In an alternative embodiment, the disposable portion 304 may be configured to simply fall from the reusable portion 302 after the cam ring 322 is moved into the fifth configuration. In a further alternative embodiment, the controller 136 may transition the cam ring 322 from the fourth configuration to the fifth configuration after a predetermined time period following actuation of the user input 312, if required. The predetermined time period may be, for example, based on the typical time period for delivering the therapeutic agent to a patient.

In any case, when the disposable portion 304 is no longer within the reusable portion 302, the controller 136 causes the actuator 316 to rotate the cam ring 322 back to the first configuration illustrated in FIG. 13. The actuator 316 may rotate the cam ring 322 in the counter clockwise direction (as viewed from the proximal end 306 of the reusable portion 302) back to the first configuration or in the clockwise direction. When the cam ring 322 is returned to the first configuration, the reusable portion 302 is ready to receive a new disposable portion 304 to repeat the above-described process to inject a subsequent dose of the therapeutic agent.

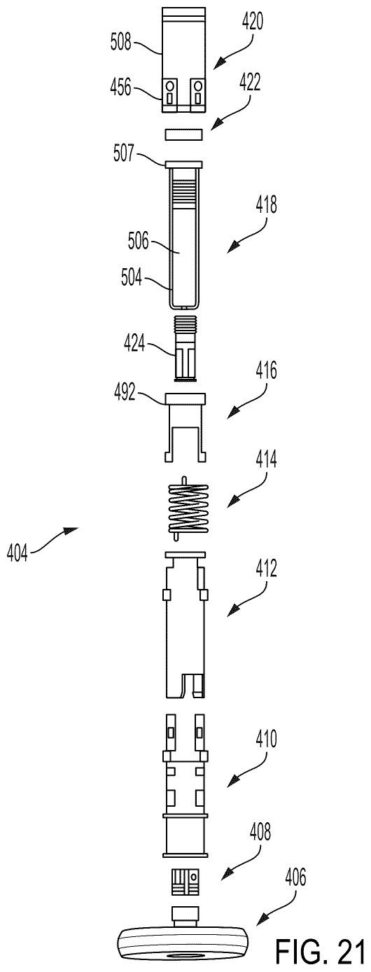

Referring now to FIGS. 21 through 33, another embodiment of a disposable portion or syringe carrier 404 for use with a motor driven therapeutic agent delivery device is illustrated. The syringe carrier 404 may be used with a reusable portion such as reusable portion 302 described above. Referring first to FIG. 21, in one embodiment, the syringe carrier 404 includes a base cap 406, an RNS puller 408, a bottom housing 410, an inject stop 412 (also referred to herein as “a first portion” or a “a lock portion”), a retraction spring 414, a lockout spacer 416, a syringe assembly 418, a top housing 420, and a spacer 422. Bottom housing 410 and the top housing 420 generally make up the housing body of the syringe carrier 404. In certain embodiments, syringe carrier 404 may be configured to accommodate different syringe assemblies 418 containing different dosages of therapeutic agent, such as 1 mL, 2 mL, etc. In such embodiments, the syringe assemblies 418 may be different sizes, which may require modifications to the lockout spacer 416, the inject stop 412 and the base cap 406. For example, in an embodiment using a 1 ml syringe assembly 418, the spacer 422 may be omitted.

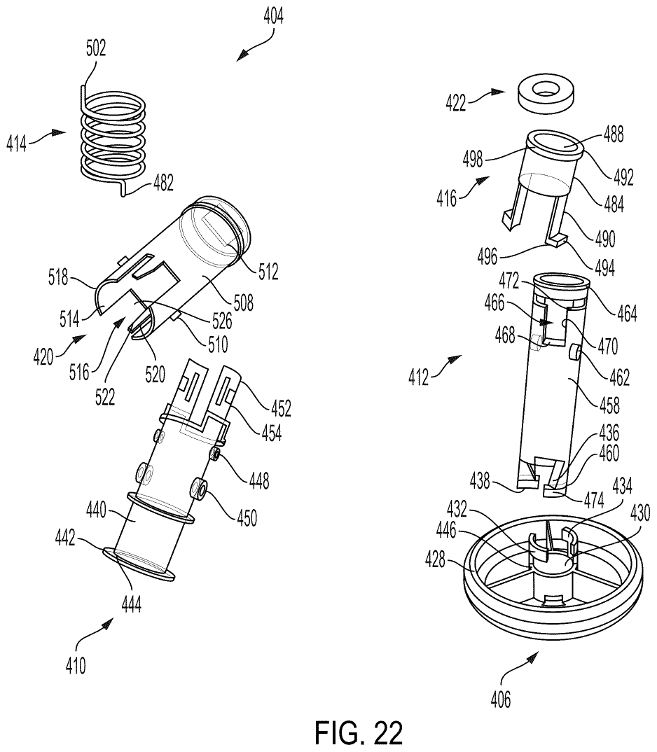

The base cap 406 receives the RNS puller 408, which engages the needle shield 424 of the syringe assembly 418 to disconnect the needle shield 424 from the syringe assembly 418, thereby exposing the needle 426 (FIG. 28), after the base cap 406 has been unlocked as described below. As best shown in FIG. 22, the base cap 406 includes a disk-shaped body 428, a central opening 430 for receiving RNS puller 408, and a pair of arms 432 extending upwardly from central opening 430. Each arm 432 includes a tab 434 which engages a notch 436 formed in the distal end 438 of the inject stop 412 as is further described below. Tab 434 may form at least partially as a base cap lock for coupling to the carrier base cap lock in a first configuration as shown in FIG. 25.

Bottom housing 410 includes a substantially cylindrical body 440 with an inner diameter that is slightly larger than an outer diameter of the inject stop 412, thereby permitting the inject stop 412 to be received within the bottom housing 410. Bottom housing 410 also includes a lower flange 442 that engages the base cap body 428 when syringe carrier 404 is assembled. When the base cap 406 is removed from the syringe carrier 404, the lower flange 442 of the bottom housing 410 is used to engage the skin of the patient to provide the proper distance for piercing the skin of the patient by needle 426 of the syringe assembly 418 as described herein. A notch 444 is formed in bottom housing 410 and sized to receive a key 446 extending upwardly from base cap body 428 to prevent twisting or rotation of the base cap body 428 as the inject stop 412 is rotated in the manner described below. Bottom housing 410 also includes a pair of upper protrusions 448 and a pair of lower protrusions 450 which engage the leading tracks 378 and securing tracks 382 of reusable portion 302 (FIG. 13) as is further described below. Lower protrusions 450 are elongated along the longitudinal axis A of syringe carrier 404 to provide extended engagement with the leading tracks 378 of the distal cylinder 374 of the reusable portion 302. Finally, bottom housing 410 also includes a pair of engagement arms 452 which are slightly resilient and include locking tabs 454 which engage with locking recesses 456 (FIG. 21) formed in the top housing 420 when the syringe carrier 404 is assembled, thereby securing the bottom housing 410 to the top housing 420.

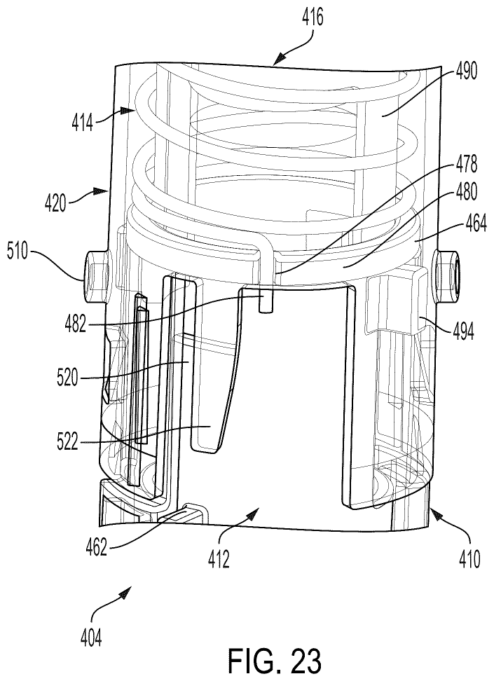

The inject stop 412 is movably coupled to the syringe carrier housing and includes an injection lock to prevent the syringe assembly from moving relative to the syringe carrier housing and a carrier base cap lock to prevent base cap from being removed from the syringe assembly until the right time as described below. Inject stop 412 generally includes a substantially cylindrical body 458 with a pair of distal arms 460, a pair of driven tabs 462, a proximal collar 464, and a pair of control recesses 466 to permit and guide movement of lockout spacer 416 in the manner described below. Each of the control recesses 466 includes a lower stop surface 468, a side stop surface 470, and an upper stop surface 472. Upper stop surface 472 may form at least partially an aspect of the injection lock. The distal arms 460 include extensions 474 extending radially that form notches 436 which receive the tabs 434 of the base cap arms 432 to retain the base cap 406 in position until inject stop 412 is rotated in the manner described below. Notches 436 may form at least partially an aspect of the carrier base cap lock. As best shown in FIG. 23, the proximal collar 464 of inject stop 412 includes a retention notch 478 formed extending axially into the outer surface 480 of the proximal collar 464 and configured to receive a first end 482 of the retraction spring 414 that has a shape to extend axially within the notch 478.

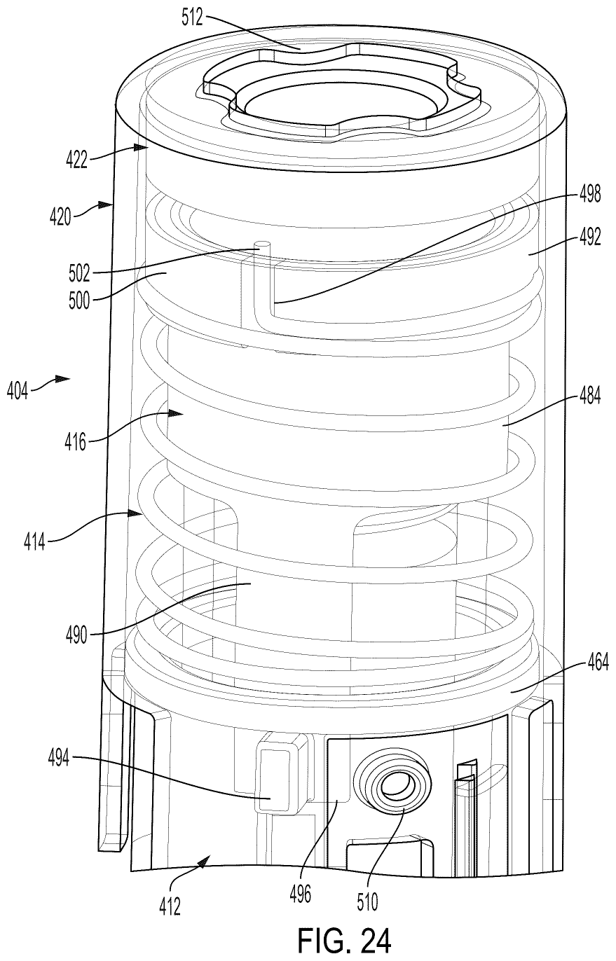

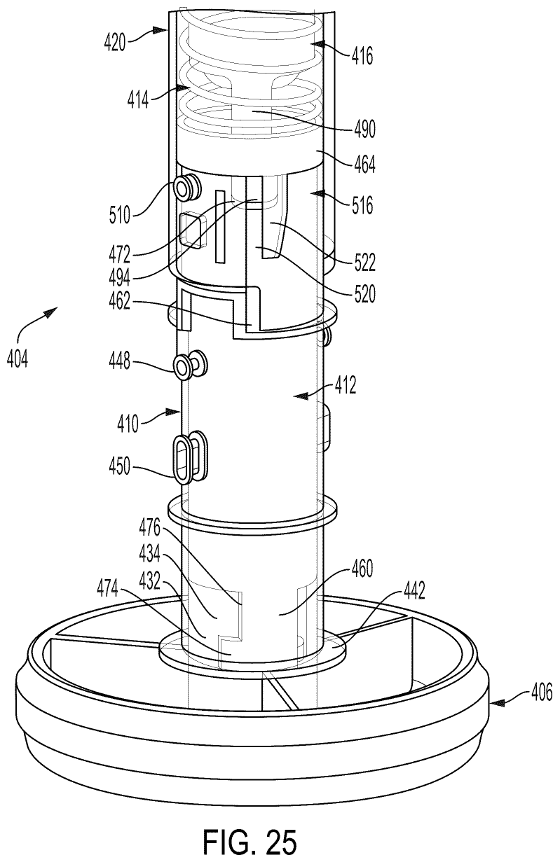

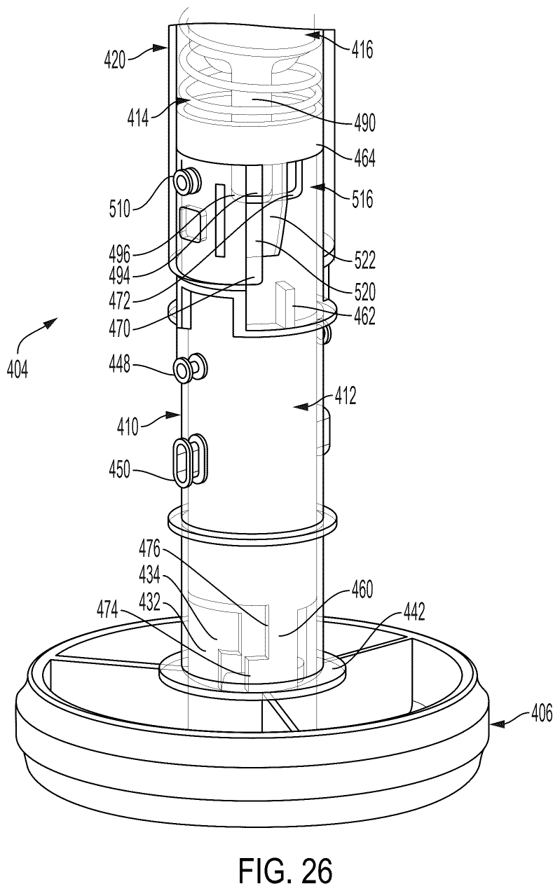

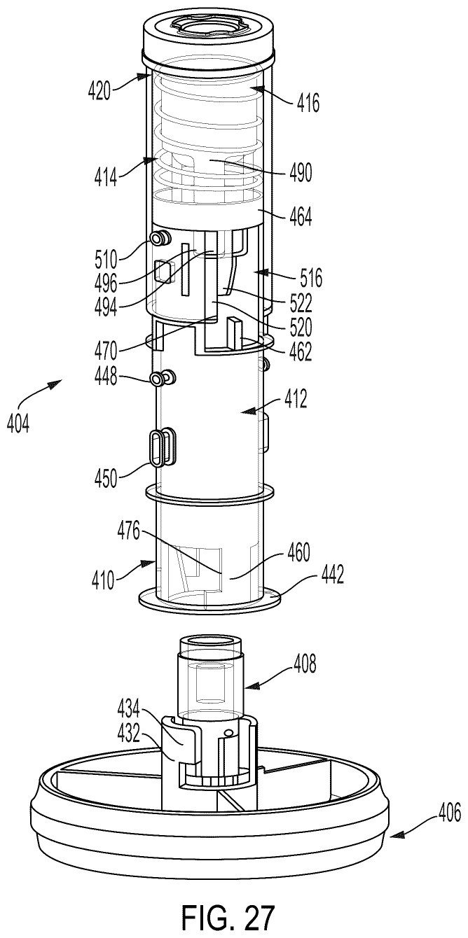

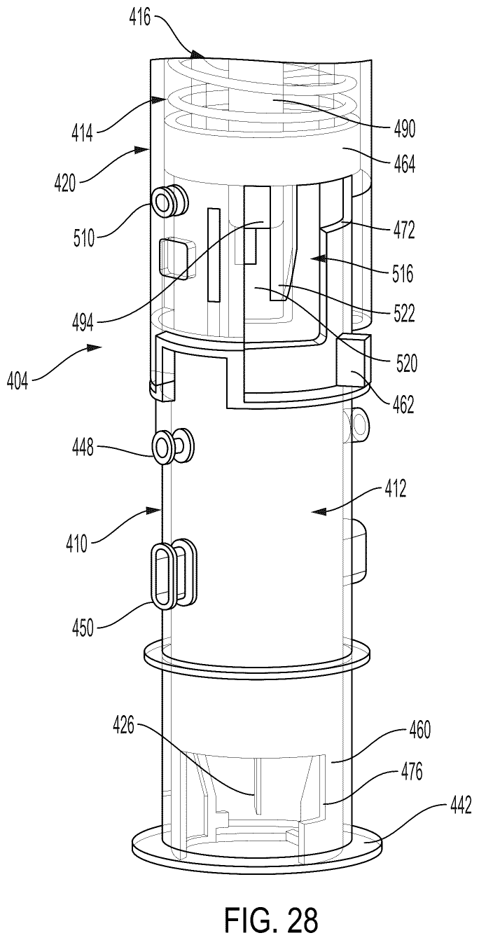

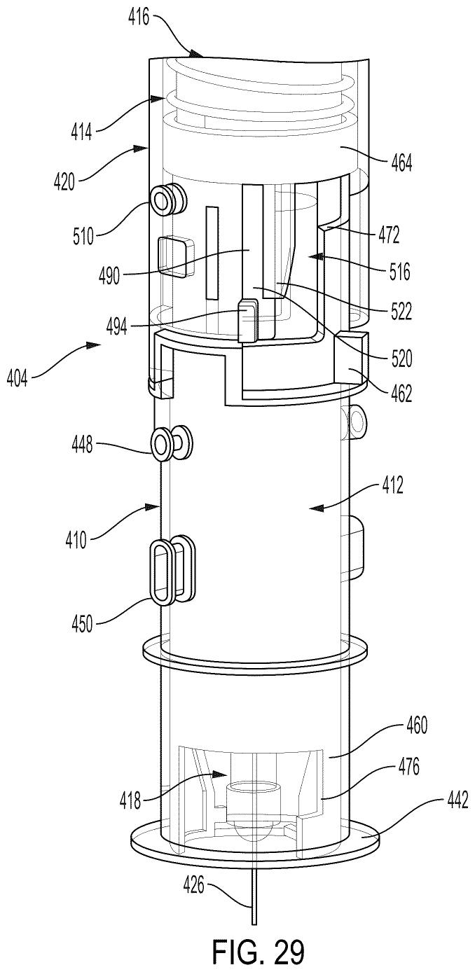

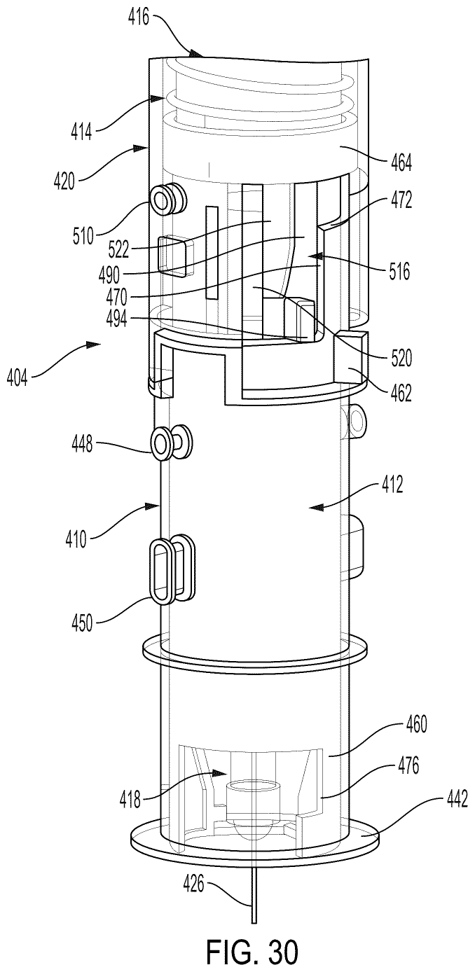

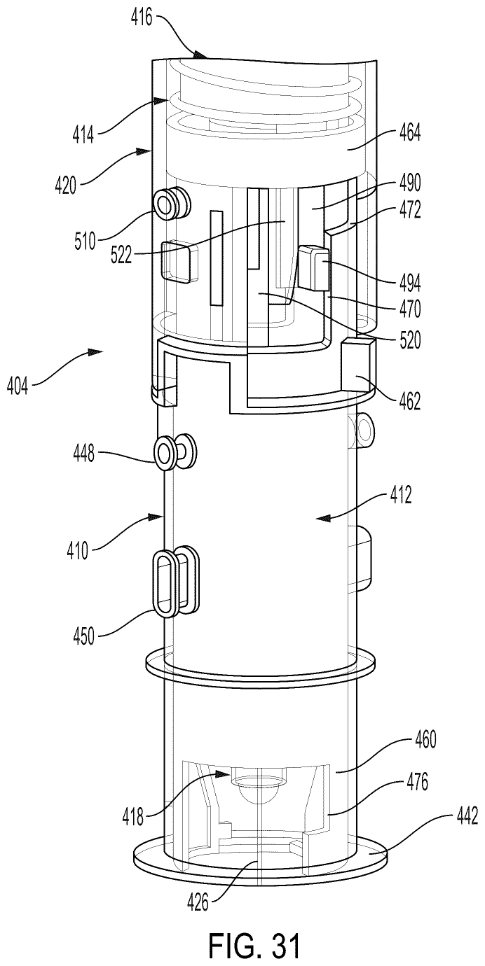

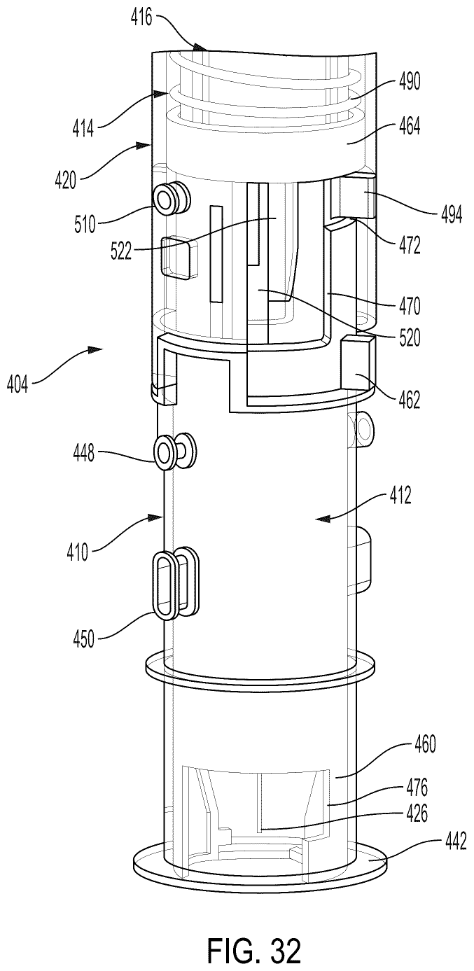

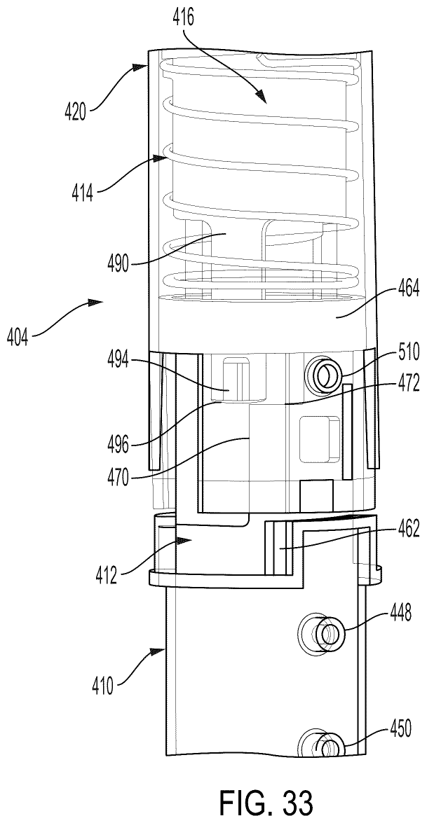

Referring back to FIG. 22, lockout spacer 416 (also referred herein to as “a lockout element”) includes a cylindrical body 484 defining a central opening 488, a pair of distal arms 490 extending from the cylindrical body 484 and a proximal flange 492. Each of the distal arms 490 includes a protrusion 494 (also referred herein to as “a lockout lock”) which extends radially outwardly from the distal arm 490. The distal arms 490 each further include a distal surface 496 which interacts with portions of the inject stop control recesses 466 in the manner described below. As best shown in FIG. 24, the proximal flange 492 includes a retention notch 498 formed extending axially into the outer surface 500 of the proximal flange 492 and configured to receive a second end 502 of the retraction spring 414 that has a shape to extend axially within the notch 498. To this end, the second end 502 can push circumferentially against a confronting wall of the notch 498 to provide the rotational movement of the spacer 416 relative to the device housing.

Still referring to FIG. 24, the retraction spring 414 is a helical torsion spring that, upon assembly of syringe carrier 404, biases lockout spacer 416 toward counter clockwise rotation relative to inject stop 412. The retraction spring 414 extends between the first end 482 (FIG. 23) which engages the retention notch 478 of the proximal collar 464 of inject stop 412 and the second end 502 which engages the retention notch 498 of the proximal flange 492 of the lockout spacer 416. Upon assembly of syringe carrier 404, the retraction spring 414 is stretched in a clockwise direction and the ends 482, 502 are inserted into retention notches 478, 498, respectively, such that the lockout spacer 416 would rotate in a counter-clockwise direction and the inject stop 412 would rotate in a clockwise direction if the parts were not prevented from rotation by other components of the syringe carrier 404 as described herein. The retraction spring 414 overlays the cylindrical body 484 of the lockout spacer 416 and has an outer diameter that is smaller than an inner diameter of top housing 420.

Referring back to FIG. 21, syringe assembly 418 includes a barrel 504 which contains therapeutic agent 506, a hollow needle 426 (FIG. 28) for injection of the therapeutic agent 506, an upper flange 507, and a needle shield 424 detachably connected to barrel 504 and configured to enclose the needle 426 to prevent accidental contact of the needle 426 with skin. As described above with reference to device 100, the needle 426 and syringe assembly 418 are moveable from a stowed configuration wherein the needle 426 is retracted within syringe carrier 404 to a deployed configuration wherein the needle 426 extends at least partially below the lower flange 442 of the bottom housing 410 to pierce the skin of the patient. In general, during the operation described below, the drive mechanism 314 and the user input 312 of the reusable portion 302 described above with reference to device 300 cause therapeutic agent 506 to be forced through needle 426 into the patient.

Still referring to FIG. 21, the spacer 422 of syringe carrier 404 is positioned on top of the upper flange 507 of the syringe assembly 418, which is positioned on top of the proximal flange 492 of lockout spacer 416. The spacer 422 has a height that accommodates the difference in length of the 1 ml syringe assembly and the 2 ml syringe assembly 418 to permit use of the same bottom housing 410, top housing 420 and retraction spring 414 for each syringe carrier design. In this manner, the spacer 422 locates the tip of the needle 426 of the 2 mL syringe assembly 418 at the same distance from the skin of the patient as the distance between the tip of the needle of the 1 ml syringe assembly when assembled into the 1 ml syringe carrier as described below. Those skilled in the art will appreciate that use of the spacer 422 permits use of the same drive mechanism of the reusable portion 302 (i.e., the same distance of travel) with both 1 ml syringe assemblies and shorter 2 ml syringe assemblies.

Referring now to FIGS. 21 and 22, top housing 420 includes a substantially cylindrical body 508 with an inner diameter that is slightly larger than an outer diameter of the inject stop 412, thereby permitting the inject stop 412 to be partially received within the top housing 420. Top housing 420 also includes a pair of protrusions 510 which engage the leading tracks 378 and securing tracks 382 of the reusable portion 302 (FIG. 13) as is further described herein. Top housing 420 also includes an upper wall 512 which compresses the spacer 422, the lockout spacer 416, the retraction spring 414 and the inject stop 412 within top housing 420 and bottom housing 410 when top housing 420 is connected to bottom housing 410 by engaging the locking tabs 454 of the engagement arms 452 of the bottom housing 410 with the locking recesses 456 (FIG. 21) formed on the inner surface 514 of the body 508 of the top housing 420. Finally, the top housing 420 also includes a pair of lockout spacer control tracks 516 formed as recesses in the distal end 518 of the top housing 420. These lockout spacer control tracks 516 control the travel of the lockout spacer 416 during needle insertion in the manner described below. Each lockout spacer control track 516 includes a retaining notch 520 formed by a control finger 522. The retaining notches 520 receive the protrusion 494 of a distal arms 490 of the lockout spacer 416 when the syringe carrier 404 is assembled. The control fingers 522 prevents counter clockwise rotation of the lockout spacer 416 by preventing movement of the protrusions 494 as is further described below. The lockout spacer control tracks 516 further include retention recesses 526 which cooperate with the control recesses 466 of the inject stop 412 to prevent subsequent needle insertion in the manner described below. Carrier lock elements may include one or more of protrusions 510, 448, 450.

After syringe carrier 404 is assembled as shown in FIG. 25, it is inserted into the reusable portion 302 for use in a manner that is substantially similar to that described above with reference to device 300. Syringe carrier 404 is inserted into reusable portion 302 in a manner similar to that depicted in FIG. 15 such that the protrusions 510 of top housing 420 are passed through the leading tracks 378 of the distal cylinder 374 and into the securing tracks 382 of the cam ring 322. The upper protrusions 448 of bottom housing 410 and the lower protrusions 450 of bottom housing 410 are positioned within the leading tracks 378 of the distal cylinder 374. As shown, the protrusions 494 of the distal arms 490 of the lockout spacer 416 are positioned within the lockout spacer control tracks 516, and more specifically within the retaining notches 520 formed by the control fingers 522 of the top housing 420. The control fingers 522 prevent the protrusions 494 (and the lockout spacer 416 generally) from rotating counter clockwise as a result of the biasing force of the retraction spring 414. Additionally, the lock portion the protrusions 494 are positioned above the upper stop surfaces 472 of the control recesses 466 of inject stop 412. In this position, the lockout spacer 416 is in an initial state or prevent state to inhibit the syringe assembly from moving relative to the carrier housing. Also, the tabs 434 of the base cap arms 432 are positioned within the notches 476 formed by the extensions 474 of the distal arms 460 of inject stop 412. When in this first configuration, base cap 406 and the RNS puller 408 cannot be removed from the syringe carrier 404.

In this configuration, the controller 136 waits for a signal from a sensor 140 disposed in the reusable portion 302 adjacent the base cap 406 of syringe carrier 404 indicating the detection and identification of syringe carrier 404. After the syringe carrier 404 is identified and verified, a first rotation of the cam ring 322 positions the protrusions 510 of the top housing 420 in the securing portions 392 of the securing tracks 382 of the cam ring 322, thereby locking the syringe carrier 404 to the reusable portion 302 in a manner similar to that depicted in FIG. 16.

Referring now to FIG. 26, the above-described rotation of cam ring 322 also causes the cam ring 322 to engage the driven tabs 462 of inject stop 412 and rotate the inject stop 412 relative to the housing body of the syringe carrier 404 such that the notches 476 formed by the extensions 474 of the distal arms 460 of inject stop 412 move out of engagement with the tabs 434 of the base cap arms 432, thereby unlocking the base cap 406. This rotation also causes the control recesses 466 (FIG. 22) of the inject stop 412 to rotate away from the prevent state such that the protrusions 494 of the distal arms 490 of the lockout spacer 416 are positioned in substantial alignment with the side stop surfaces 470 of the control recesses 466. It should be noted, however, that the distal surfaces 496 of the distal arms 490 remain in engagement with the upper stop surfaces 472 of the control recesses 466 of the inject stop 412. Thus, when in the second configuration shown in FIG. 26, the base cap 406 and RNS puller 408 may be pulled downwardly out of the reusable portion 302 to remove the needle shield 424 of the syringe assembly 418. This pulling force on the syringe assembly 418 cannot, however, result in movement of the syringe assembly 418 because the distal surfaces 496 of the distal arms 490 of the lockout spacer 416 are still engaged with the upper stop surfaces 472 of the control recesses 466. In other words, the syringe assembly 418 cannot move downwardly because the lockout spacer 416 cannot move downwardly. Since the lockout spacer 416 cannot be moved downwardly, the design prevents tripping the single-use lockout described below when removing the base cap 406. FIG. 27 depicts base cap 406 and RNS puller 408 after removal from the reusable portion 302.

Referring now to FIG. 28, after the base cap 406 and RNS puller 408 are removed, the controller 136 waits for detection of the patient's skin. More specifically, the controller 136 waits for a signal from a sensor 140 positioned in the reusable portion 302 adjacent the lower flange 442 of bottom housing 410. For example, a skin sensor 140 may be a capacitance or other sensor configured to detect contact with the skin of the patient and provide a sensed signal to the controller 136 that changes with contact to indicate such contact. As an optional next step, the controller 136 may wait for a signal from another sensor 140 operatively coupled to an unlock input (not shown), such as a slider or wiper, that is actuated by the user. When the patient's skin is detected (and optionally when the user has actuated the unlock input), the reusable portion 302 may provide an indication to the user that the device is enabled for needle injection. The indication may be visual (e.g., illumination of the user input 312 or displaying a message to the user on a display, or both) or audible (e.g., playing a message over a speaker to the user).

Also, when the patient's skin is detected (and optionally when the user has actuated the unlock input), the drive mechanism 314 causes the cam ring 322 to rotate further in the counter clockwise direction, thereby further engaging the driven tabs 462 of inject stop 412 and causing inject stop 412 to rotate to the position shown in FIG. 28. In this third configuration, the lockout spacer 416 is no longer engaged with the upper stop surfaces 472 of the control recesses 466 of the inject stop 412.

When the user actuates the user input 312, the drive mechanism 314 of the reusable portion 302 moves the lockout spacer 416 and the syringe assembly 418 downwardly such that the needle 426 of the syringe assembly 418 pierces the skin of the patient. The drive mechanism 314 then further causes the delivery of the therapeutic agent 506 through the needle 426 in the manner described herein. If the user input 312 is not actuated, or not actuated within a predetermined time limit (i.e., a dose of therapeutic agent 506 is not delivered to the patient within the predetermined time limit), then the actuator 316 may cause the cam ring 322 to rotate to eject the syringe carrier 404. The skin sensor 140 described above may continue monitoring contact the patient's skin during the needle insertion and dose delivery processes.

As the drive mechanism 314 causes distal movement of the lockout spacer 416 and the syringe assembly 418 relative to the housing body of the syringe carrier 404, the protrusions 494 of distal arms 490 of the lockout spacer 416 are moved to the position shown in FIG. 29. In this fourth configuration, the retraction spring 414 is compressed and the protrusions 494 of the distal arms 490 have moved out of the retaining notches 520 and are below the control fingers 522 of the of the lockout spacer control tracks 516 of the top housing 420. Retraction spring 414 when loaded may be configured to provide an axial force and torque force. As such, lockout spacer 416 is free to rotate in the counter-clockwise direction under the biasing force of retraction spring 414 to begin the movement to the single-use lockout state described below. In this fourth configuration, the needle 426 and the syringe assembly 418 are shown in the deployed configuration to pierce the skin of a patient for delivery of the therapeutic agent 506. Notice the protrusion 494 moves distally relative to the inject stop from a proximal start position to an injection position.

FIG. 30 depicts the first segment of rotation of the lockout spacer 416. As shown, the protrusions 494 of the distal arms 490 of the lockout spacer 416 have rotated into engagement with the side stop surfaces 470 of the control recesses 466 of the inject stop 412. After delivery of the therapeutic agent 506 is complete, the syringe assembly 418 and the inject stop 412 are moved proximally relative to the housing body of the syringe carrier 404 by force supplied by the retraction spring 414. In this process, the inject stop 412 continues to rotate relative to the housing body of the syringe carrier 404 in the counter clockwise direction by torque force supplied by the retraction spring 414. Notice the protrusion 494 moves angularly relative to the inject stop from the injection position to a retractable position.

FIG. 31 depicts lockout spacer 416 and syringe assembly 418 approximately mid-way through the proximal movement described above as the protrusions move from its retractable position. Here, the protrusion 494 moves proximally relative to the inject stop from the retractable position toward a lockout position. FIG. 32 depicts the syringe carrier 404 in a fifth configuration. In this fifth configuration, lockout spacer 416 has been retracted proximally relative to the housing body of the syringe carrier 414 by retraction spring 414 and rotated counter-clockwise under the biasing force of the retraction spring 414 such that the protrusions 494 of the distal arms 490 of the lockout spacer 416 are positioned further angularly in the control recesses 466 of the inject stop 412 in contact with the upper stop surfaces 472 at its final lockout position. Additionally, the syringe assembly 418 is fully retracted and in the stowed configuration. In this position, the syringe carrier 404 is in the single-use lockout state. The lockout spacer 416 (and therefore the syringe assembly 418 and the needle 426) cannot be moved downwardly again because of the engagement between the protrusions 494 and the upper stop surfaces of the inject stop 412. The retraction spring 414 biases the lockout spacer 416 in the lockout state.

When the user actuates an eject slider or other input (not shown), the cam ring 322 rotates relative to the distal cylinder 374 (in the same direction illustratively, in the counter-clockwise direction as viewed from the proximal end 306 of reusable portion 302) such that the protrusions 510 of the top housing 420 are disposed in the egress portions 394 of the securing tracks 382 of the cam ring 322 in a manner substantially as described above with reference to FIG. 18. As a result, the cam ring 322 pushes the protrusions 510 and the syringe carrier 404 distally to position the syringe carrier 404 for ejection. FIG. 33 depicts syringe carrier 404 after cam ring 322 has rotated syringe carrier 404 as described above. It should be noted syringe carrier 404 remains in the single-use lockout state because the distal surfaces 496 of the distal arms 490 of the lockout spacer 416 remain engaged with the upper stop surfaces 472 of the control recesses 466.

It is understood that protrusion and track arrangements of associated components specifically described herein may be switched to the opposite arrangement such that the component shown with the protrusion may be configured with the track and vice versa. Although devices are described as a reusable portion and a syringe carrier that is insertable into the reusable portion and removable from the reusable portion for disposable, it is understood that the reusable portion may be disposable after a single use and/or the syringe carrier may be permanently attached to the drive portion (referred to herein as the reusable portion) to define a fully integrated disposable device after contents of the syringe carrier is at least partially exhausted.

While this invention has been shown and described as having preferred embodiments, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

Various aspects are described in the description in this disclosure, which include, but are not limited to, the following aspects:
1. A therapeutic agent delivery device, comprising: a reusable portion, comprising: a first housing; a drive mechanism; and a cam ring rotatably movable in the first housing by the drive mechanism; and a syringe carrier, comprising: a second housing including a first protrusion, the first protrusion entering a securing track of the cam ring upon insertion of the syringe carrier into the reusable portion; a first portion rotatably coupled to the second housing; a syringe assembly including a barrel containing a therapeutic agent, a needle, and a needle shield detachably coupled to the barrel, the syringe assembly being movable between a stowed configuration and a deployed configuration wherein the needle extends from the second housing; and a base cap including a first surface engageable with the first portion to lock the base cap to the first portion; wherein a first rotation of the cam ring rotates a securing portion of the securing track to receive the first protrusion to lock the syringe carrier to the reusable portion and rotates the first portion to disengage the first surface of the base cap from the first portion to unlock the base cap from the first portion.
2. The therapeutic agent delivery device of aspect 1, wherein the first portion is a locking clip coupled to the second housing and movable within a recess formed in the second housing.
3. The therapeutic agent delivery device of aspect 2, wherein the base cap further includes a rigid needle shield puller extending into the second housing, the rigid needle shield puller including an arm forming the first surface engageable with an inner protrusion formed on the locking clip that extends through a slot formed in the second housing recess.
4. The therapeutic agent delivery device of aspect 2, wherein the locking clip includes an outer protrusion that is engaged by the securing track to rotate the locking clip during the first rotation.
5. The therapeutic agent delivery device of any one of aspects 1-4, wherein the first portion is an inject stop disposed within the second housing and rotatable relative to the second housing.
6. The therapeutic agent delivery device of aspect 5, wherein the base cap further includes a tab forming the first surface, the tab configured to engage with a notch formed on the inject stop to lock the base cap to the inject stop.
7. The therapeutic agent delivery device of aspect 5, wherein the inject stop includes a driven tab that is engaged by the securing track to rotate the inject stop during the first rotation.
8. The therapeutic agent delivery device of aspect 5, wherein the syringe carrier further comprises a lockout spacer including a second protrusion and being movable from an initial state to a single use lockout state.
9. The therapeutic agent delivery device of aspect 8, wherein the inject stop includes a control recess configured to receive the second protrusion, the control recess including an upper stop surface that is engaged with the second protrusion when the lockout spacer is in the initial state and the single use lockout state, thereby preventing the syringe assembly from moving from the stowed configuration to the deployed configuration.
10. The therapeutic agent delivery device of aspect 9, wherein the syringe carrier further comprises a retraction spring connected between the lockout spacer and the inject stop to bias the lockout spacer for rotation toward the single use lockout state.
11. The therapeutic agent delivery device of aspect 10, wherein when in the initial state, the lockout spacer is prevented from moving toward a distal end of the second housing by the upper stop surface and prevented from rotating toward the single use lockout state by engagement between the second protrusion and a control finger of the second housing.
12. The therapeutic agent delivery device of aspect 10, wherein in response to a second rotation of the cam ring the inject stop is rotated to disengage the upper stop surface of the control recess from the second protrusion of the lockout spacer, thereby permitting the syringe assembly to move from the stowed configuration to the deployed configuration for delivery of the therapeutic agent.
13. The therapeutic agent delivery device of aspect 12, wherein as the syringe assembly is moved to the deployed configuration, the second protrusion is moved out of engagement with the control finger of the second housing.
14. The therapeutic agent delivery device of aspect 13, wherein during movement of the syringe assembly to the stowed configuration after delivery of the therapeutic agent, the retraction spring rotates the lockout spacer to the single use lockout state wherein the second protrusion engages the upper stop surface.
15. The therapeutic agent delivery device of any one of aspects 1-14, wherein the first housing further comprises a leading track and the second housing further comprises a second protrusion, the second protrusion entering the leading track upon insertion of the syringe carrier into the reusable portion and remaining in the leading track during the first rotation.
16. The therapeutic agent delivery device of any one of aspects 1-15, wherein the base cap includes a tab that is engaged with a key formed in the second housing to prevent rotation of the base cap.
17. The therapeutic agent delivery device of any one of aspects 1-16, wherein the securing track further includes an egress portion extending helically along an inner surface of the cam ring.
18. The therapeutic agent delivery device of aspect 17, wherein a second rotation of the cam ring causes the first protrusion to travel within the egress portion, thereby unlocking the syringe carrier from the reusable portion.
19. A therapeutic agent delivery device, comprising: a reusable portion, comprising: a first housing; a drive mechanism; and a cam ring rotatably movable in the first housing by the drive mechanism; and a syringe carrier, comprising a second housing including a first protrusion and a second protrusion, the first protrusion entering a securing track of the cam ring upon insertion of the syringe carrier into the reusable portion; a lockout spacer including a pair of third protrusions and being movable from an initial state to a single use lockout state; an inject stop including a pair of control recesses for receiving the pair of third protrusions, and a pair notches; a retraction spring connected to bias the lockout spacer for rotation toward the single use lockout state; a syringe assembly including a barrel containing a therapeutic agent, a needle, and a needle shield detachably coupled to the barrel, the syringe assembly being movable between a stowed configuration and a deployed configuration wherein the needle extends from the second housing; and a base cap including a pair of tabs configured to engage the pair of notches to lock the base cap to the inject stop; wherein a first rotation of the cam ring rotates a securing portion of the securing track to receive the first protrusion to prevent removal of the syringe carrier, the first rotation further rotating the inject stop to place the syringe carrier in a first configuration wherein the pair of tabs disengage from the pair of notches to unlock the base cap for removal, and the pair of third protrusions engage upper stop surfaces of the pair of control recesses to prevent movement of the syringe assembly toward the deployed configuration as the base cap is removed to detach the needle shield from the barrel; wherein a second rotation of the cam ring rotates the inject stop to place the syringe carrier in a second configuration wherein the pair of third protrusions disengage from the upper stop surfaces to permit movement of the syringe assembly to the deployed configuration for delivery of the therapeutic agent; and wherein during movement of the syringe assembly to the stowed configuration after delivery of the therapeutic agent, the retraction spring rotates the lockout spacer to the single use lockout state wherein the pair of third protrusions engage the upper stop surfaces.
20. The therapeutic agent delivery device of aspect 19, wherein after the syringe assembly is moved to the deployed configuration, the retraction spring causes the lockout spacer to rotate in the first direction until the pair of third protrusions engage side stop surfaces of the pair of control recesses.
21. A method for delivering a therapeutic agent using a delivery device including a reusable portion and a syringe carrier, comprising: inserting the syringe carrier into a housing of the reusable portion such that a first protrusion of the syringe carrier enters a securing track of a cam ring of the reusable portion; activating a drive mechanism to cause a first rotation of the cam ring, the first rotation rotating a securing portion of the securing track to receive the first protrusion to lock the syringe carrier to the reusable portion, the first rotation further causing rotation of an inject stop of the syringe carrier to disengage an engagement surface of a base cap of the syringe carrier to unlock the base cap from the inject stop; and activating the drive mechanism to cause a second rotation of the cam ring, the second rotation rotating the inject stop to disengage a second protrusion of a lockout spacer from a stop surface of the inject stop to permit movement of a syringe assembly of the syringe carrier to move to a deployed configuration for delivery of the therapeutic agent; wherein during movement of the syringe assembly to a stowed configuration after delivery of the therapeutic agent, a retraction spring of the syringe carrier rotates the lockout spacer to a single use lockout state wherein the second protrusion engages the stop surface of the inject stop.
22. A therapeutic agent delivery device, comprising: a reusable portion, comprising: a first housing; a drive mechanism; and a lock actuator movable in the first housing by the drive mechanism; and a syringe carrier, comprising: a carrier housing including a first carrier lock element coupled to a corresponding lock actuator element of the lock actuator upon insertion of the syringe carrier into the reusable portion, a first portion movably coupled to the carrier housing; a syringe assembly configured to hold a medication; a base cap including a first surface configured to engage with the first portion to couple the base cap to the first portion; wherein in response to a first movement of the lock actuator a securing portion of the lock actuator element is moved to receive the first carrier lock element to lock the syringe carrier to the reusable portion, and the first portion is moved to disengage the first surface of the base cap from the first portion to unlock the base cap from the first portion.
23. The therapeutic agent delivery device of aspect 22, wherein the first portion is a locking clip movable within a recess formed in the carrier housing.
24. The therapeutic agent delivery device of aspect 22, wherein the first portion is an inject stop disposed within the carrier housing and rotatable relative to the carrier housing.
25. The therapeutic agent delivery device of aspect 24, wherein the base cap further includes a tab forming the first surface, the tab configured to engage with a notch formed on the inject stop to lock the base cap to the inject stop.
26. The therapeutic agent delivery device of any one of aspects 22-25, wherein the syringe carrier further comprises a lockout spacer including a protrusion and being movable from an initial state to a single use lockout state, wherein the inject stop includes a control recess configured to receive the protrusion, the control recess including an upper stop surface that is engaged with the protrusion when the lockout spacer is in the initial state and the single use lockout state, thereby preventing the syringe assembly from moving from a stowed configuration to a deployed configuration.
27. The therapeutic agent delivery device of aspect 26, wherein the syringe carrier further comprises a retraction spring connected between the lockout spacer and the inject stop to bias the lockout spacer for rotation toward the single use lockout state.\
28. The therapeutic agent delivery device of any one of aspects 22-27, wherein a second movement of the lock actuator moves the first carrier lock element into an egress portion of the lock actuator element to unlock the syringe carrier from the reusable portion.
29. A syringe carrier, comprising: a carrier housing; a lock portion movably coupled to the carrier housing, the lock portion having an injection lock and a carrier base cap lock; a syringe assembly configured to hold a medication; a lockout element axially fixed relative to the syringe assembly, the lockout element having a lockout lock, wherein the lockout lock is coupled to the injection lock in a prevent state to prevent movement of the syringe assembly relative to the carrier housing; and a base cap having a base cap lock, the base cap lock coupled to the carrier base cap lock in a first configuration, and the base cap lock is decoupled from the carrier base cap lock in a second configuration to permit removal of the base cap from the syringe assembly; wherein, in response to a first movement of the lockout element, the base cap lock and the carrier base cap lock is transitioned from the first configuration to the second configuration, and, in response to a second movement of the lockout element, the lockout lock is moved relative to the injection lock away from the prevent state to allow relative movement between the syringe carrier and the carrier housing.
30. The syringe carrier of aspect 29, wherein after the second movement the lockout element and the syringe assembly are distally movable relative to the carrier housing to an injection position.
31. The syringe carrier of any one of aspects 29-30, wherein during and/or after the second movement the lockout element is moved angularly relative to the carrier housing.
32. The syringe carrier of aspect 31, wherein during and/or after the second movement the lockout element is moved angularly relative to the carrier housing to a retractable position, wherein after the lockout element is moved the retractable position, the lockout element is moved angularly relative to the carrier housing to a lockout position, wherein the lockout lock is coupled to the injection lock in the prevent state.
33. The syringe carrier of aspect 31, wherein when the lockout element is at the retractable position, the lockout element and the syringe assembly are proximally movable relative to the carrier housing.
34. The syringe carrier of any one of aspects 29-33, further comprising a spring coupled between the lockout element and the lock portion, the spring configured to provide a rotational force to the lockout element to allow the lockout element to move to the retractable position.
35. The syringe carrier of aspect 34, wherein the spring is configured to provide an axial force to move the lockout element and the syringe assembly proximally to a retracted syringe position.
36. The syringe carrier of aspect 34, wherein the spring is configured to provide a rotational force to the lockout element to allow the lockout element to move to the lockout position.
37. A therapeutic agent delivery device, comprising: a reusable portion including a first housing; a drive mechanism; and a lock actuator movable relative to the first housing by the drive mechanism; and the syringe carrier of any one of aspects 29-36, wherein the lock actuator is configured to move the lockout element to the first movement and the second movement after the first movement.
38. The therapeutic agent delivery device of aspect 37, wherein the syringe assembly comprises a reservoir comprising the medication.
39. A method for delivering a therapeutic agent using a delivery device including a reusable portion and a syringe carrier, comprising: providing the syringe carrier inside of a housing of the reusable portion; activating a drive mechanism to cause a first rotation of a lock actuator, the first rotation rotating a securing portion of the lock actuator to receive a first protrusion of the syringe carrier to lock the syringe carrier to the reusable portion, the first rotation further causing rotation of a lock portion of the syringe carrier to disengage a base cap lock of a base cap of the syringe carrier to unlock the base cap from the lock portion; and activating the drive mechanism to cause a second rotation of the lock actuator after the base cap has been removed from the syringe assembly, the second rotation rotating the lock portion to disengage a second protrusion of a lockout element from an injection lock of the lock portion to permit movement of a syringe assembly of the syringe carrier to move to a deployed configuration for delivery of the therapeutic agent; wherein during movement of the syringe assembly to a stowed configuration after delivery of the therapeutic agent, the lockout element rotates to a single use lockout state to prevent the syringe assembly from moving distally relative to the syringe carrier.
40. A method of operating a fluid delivery device including a reusable portion and a fluid carrier, comprising: providing the fluid carrier inside of a housing of the reusable portion; rotating a lock actuator for a first rotation with a drive mechanism of the reusable portion, the first rotation rotating a securing portion of the lock actuator to receive a first protrusion of the fluid carrier to lock the fluid carrier to the reusable portion, the first rotation further causing rotation of a lock portion of the fluid carrier to disengage a cap lock of a cap of the fluid carrier to unlock the cap from the lock portion; rotating the lock actuator for a second rotation with the drive mechanism after the cap has been removed from the fluid assembly, the second rotation rotating the lock portion to disengage a second protrusion of a lockout element from a first lock of the lock portion; and moving a fluid assembly of the fluid carrier to move to a deployed configuration for delivery of the fluid.
41. The method of aspect 40, further comprising moving the fluid assembly of the fluid carrier to a stowed configuration after delivery of the fluid, the lockout element is rotated to a single use lockout state to prevent the fluid assembly from moving distally relative to the fluid carrier.

1. A therapeutic agent delivery device, comprising:
a reusable portion, comprising:
a first housing;
a drive mechanism; and
a cam ring rotatably movable in the first housing by the drive mechanism; and
a syringe carrier, comprising:
a second housing including a first protrusion, the first protrusion entering a securing track of the cam ring upon insertion of the syringe carrier into the reusable portion;
a first portion rotatably coupled to the second housing;
a syringe assembly including a barrel containing a therapeutic agent, a needle, and a needle shield detachably coupled to the barrel, the syringe assembly being movable between a stowed configuration and a deployed configuration wherein the needle extends from the second housing; and
a base cap including a first surface engageable with the first portion to lock the base cap to the first portion;
wherein a first rotation of the cam ring rotates a securing portion of the securing track to receive the first protrusion to lock the syringe carrier to the reusable portion and rotates the first portion to disengage the first surface of the base cap from the first portion to unlock the base cap from the first portion.

2. The therapeutic agent delivery device of claim 1, wherein the first portion is a locking clip coupled to the second housing and movable within a recess formed in the second housing.

3. The therapeutic agent delivery device of claim 2, wherein the base cap further includes a rigid needle shield puller extending into the second housing, the rigid needle shield puller including an arm forming the first surface engageable with an inner protrusion formed on the locking clip that extends through a slot formed in the second housing recess.

4. The therapeutic agent delivery device of claim 2, wherein the locking clip includes an outer protrusion that is engaged by the securing track to rotate the locking clip during the first rotation.

5. The therapeutic agent delivery device of claim 1, wherein the first portion is an inject stop disposed within the second housing and rotatable relative to the second housing.

6. The therapeutic agent delivery device of claim 5, wherein the base cap further includes a tab forming the first surface, the tab configured to engage with a notch formed on the inject stop to lock the base cap to the inject stop.

7. The therapeutic agent delivery device of claim 5, wherein the inject stop includes a driven tab that is engaged by the securing track to rotate the inject stop during the first rotation.

8. The therapeutic agent delivery device of claim 5, wherein the syringe carrier further comprises a lockout spacer including a second protrusion and being movable from an initial state to a single use lockout state.

9. The therapeutic agent delivery device of claim 8, wherein the inject stop includes a control recess configured to receive the second protrusion, the control recess including an upper stop surface that is engaged with the second protrusion when the lockout spacer is in the initial state and the single use lockout state, thereby preventing the syringe assembly from moving from the stowed configuration to the deployed configuration.

10. The therapeutic agent delivery device of claim 9, wherein the syringe carrier further comprises a retraction spring connected between the lockout spacer and the inject stop to bias the lockout spacer for rotation toward the single use lockout state.

11. The therapeutic agent delivery device of claim 10, wherein when in the initial state, the lockout spacer is prevented from moving toward a distal end of the second housing by the upper stop surface and prevented from rotating toward the single use lockout state by engagement between the second protrusion and a control finger of the second housing.

12. The therapeutic agent delivery device of claim 10, wherein in response to a second rotation of the cam ring the inject stop is rotated to disengage the upper stop surface of the control recess from the second protrusion of the lockout spacer, thereby permitting the syringe assembly to move from the stowed configuration to the deployed configuration for delivery of the therapeutic agent.

13. The therapeutic agent delivery device of claim 12, wherein as the syringe assembly is moved to the deployed configuration, the second protrusion is moved out of engagement with the control finger of the second housing.

14. The therapeutic agent delivery device of claim 13, wherein during movement of the syringe assembly to the stowed configuration after delivery of the therapeutic agent, the retraction spring rotates the lockout spacer to the single use lockout state wherein the second protrusion engages the upper stop surface.

15. The therapeutic agent delivery device of claim wherein the first housing further comprises a leading track and the second housing further comprises a second protrusion, the second protrusion entering the leading track upon insertion of the syringe carrier into the reusable portion and remaining in the leading track during the first rotation.

16. The therapeutic agent delivery device of claim 1, wherein the base cap includes a tab that is engaged with a key formed in the second housing to prevent rotation of the base cap.

17. The therapeutic agent delivery device of claim 1, wherein the securing track further includes an egress portion extending helically along an inner surface of the cam ring.

18. The therapeutic agent delivery device of claim 17, wherein a second rotation of the cam ring causes the first protrusion to travel within the egress portion, thereby unlocking the syringe carrier from the reusable portion.

19-28. (canceled)

29. A syringe carrier, comprising:
a carrier housing;
a lock portion movably coupled to the carrier housing, the lock portion having an injection lock and a carrier base cap lock;
a syringe assembly configured to hold a therapeutic agent;
a lockout element axially fixed relative to the syringe assembly, the lockout element having a lockout lock, wherein the lockout lock is coupled to the injection lock in a prevent state to prevent movement of the syringe assembly relative to the carrier housing; and
a base cap having a base cap lock, the base cap lock coupled to the carrier base cap lock in a first configuration, and the base cap lock is decoupled from the carrier base cap lock in a second configuration to permit removal of the base cap from the syringe assembly;
wherein, in response to a first movement of the lockout element, the base cap lock and the carrier base cap lock is transitioned from the first configuration to the second configuration, and, in response to a second movement of the lockout element, the lockout lock is moved relative to the injection lock away from the prevent state to allow relative movement between the syringe carrier and the carrier housing.

30-36. (canceled)

37. A therapeutic agent delivery device, comprising: a reusable portion including a first housing; a drive mechanism; and a lock actuator movable relative to the first housing by the drive mechanism; and the syringe carrier of claim 29, wherein the lock actuator is configured to move the lockout element to the first movement and the second movement after the first movement.

38-41. (canceled)
